(19) United States
(12) Patent Application Publication
(10) Pub. No.: US 2025/0114299 A1
(43) Pub. Date: Apr. 10, 2025

(54) COMBINATION THERAPY INTRAVAGINAL RINGS

(71) Applicant: The population Council, Inc., New York, NY (US)

(72) Inventors: Kyle Robert Kleinbeck, Milwaukee, WI (US); Patrick Lawrence Spence, Madison, AL (US); Jeremy Peter Nuttall, Newtown Square, PA (US); Bashir Ramzanali Hansraj, Alton (GB); Bindi Ajit Dangi, Ellicott City, MD (US); Wouter de Graaff, Malmö (SE); Susan Margaret Fetherston, Belfast, Co. (GB); Johannes Antonius Hendrikus Van Laarhoven, Malmo (SE)

(21) Appl. No.: 18/729,979

(22) PCT Filed: Jan. 19, 2023
(86) PCT No.: PCT/US2023/011131

§ 371 Date: Jul. 18, 2024

Related U.S. Application Data
(60) Provisional application No. 63/301,588, filed on Jan. 21, 2022.

(51) Int. Cl. A61K 9/00; A61F 6/08; A61K 31/505; A61K 31/567

(52) U.S. Cl. CPC A61K 9/0036; A61F 6/08; A61K 31/505; A61K 31/567

(57) ABSTRACT

The present disclosure provides improved intravaginal drug delivery devices, i.e., intravaginal rings, useful for the prophylactic administration of dapivirine in combination with either an antimicrobial compound or a contraceptive to a human. The present disclosure also provides methods of blocking DNA polymerization by an HIV reverse transcriptase enzyme, methods of preventing HIV infection in a female human, methods of treating HIV infection in a female human, methods of preventing unintended pregnancy in a female human, and methods of preparing intravaginal rings.

### RELATED APPLICATION

This application claims the benefit of priority to U.S. Provisional Application No. 63/301,588, filed on Jan. 21, 2022. The entire contents of the foregoing application are incorporated herein by reference.

### BACKGROUND

The AIDS epidemic continues to exact a devastating toll on the health, economic and political infrastructure, and social fabric of communities worldwide. During 2011, almost 2.5 million people became newly infected with HIV bringing the total number of people living with HIV to an estimated 34.0 million. In the same year, almost 1.7 million people died from AIDS, raising the global death toll to over 35 million since the first cases of AIDS were identified in 1981 (UNAIDS and WHO 2011 AIDS Epidemic Update; December 2011). HIV/AIDS has become the fourth biggest cause of global mortality. Over 95 percent of new infections are occurring in developing countries, where increasing numbers of new HIV infections threaten the sustainability of expanded access to HIV treatment. Developing safe and effective HIV prevention technologies that can be made easily accessible in developing countries is, thus, an urgent public health priority.

Epidemiologic data published in the latest UNAIDS report show that women and girls bear a severe and increasingly heavy burden of the HIV epidemic. In Eastern Europe and Central Asia, an estimated 26% of adults living with HIV in 2007 were women aged 15 years or older, compared with 23% in 2001. In sub-Saharan Africa, women comprised 61% of HIV-infected adults, and among young people (aged 15-24 years) the ratio of infection had risen to three women for every man (UNAIDS and WHO 2007 AIDS Epidemic Update; December 2007).

Each year, there are also 80 million unintended pregnancies, and 358,000 women die annually from pregnancy-related complications. More than 200 million women in developing countries lack access to contraception. Accordingly, there is a strong need for developing combination products that can prevent both HIV infection and unintended pregnancy in females.

Unprotected heterosexual intercourse is currently the leading mode of HIV infection and unintended pregnancy among females. Correct and consistent use of latex condoms is one proven method of preventing HIV transmission and unintended pregnancy; however, condoms are widely regarded as inadequate prevention options for women if they are unable to negotiate condom use for fear of abuse or accusations of infidelity. Additionally, women who have sex with men in exchange for gifts or money may be reluctant to use condoms if the men are willing to pay more for sex without a condom. The female condom has been marketed as an alternative barrier method, but this device is relatively costly and requires a certain level of skill, as well as acceptance by the male partner. Developing HIV and pregnancy prevention options that women can use with or without their partner's knowledge is a pressing global concern, especially given the rapidly growing HIV infection rate among women and the absence of an effective vaccine.

Topical microbicides that can be self-administered to the vagina are one such promising alternative. Multiple clinical trials with various microbicides have been completed or are currently underway, most of which involve microbicides in gel formulation delivered via a single use applicator used prior to coitus. In order for a microbicide to be effective, it is essential that it be used correctly. Therefore it is important that a microbicide is acceptable to users, and it is likely that products that can be used less frequently will be more acceptable and will achieve better user adherence. Additionally, combination products that could prevent both HIV transmission and unintended pregnancy would be more acceptable to users.

Vaginal rings that need only be replaced at relatively long intervals may therefore have benefits over other dosage forms that must be used more frequently. Ring-shaped devices for the controlled administration of steroid substances (substantially water-insoluble drugs) into the vagina have been developed, such as Estring®, Femring® and Nuvaring®. There are two basic types of vaginal rings: reservoir rings and matrix rings.

A reservoir ring comprises a full or partial-length core loaded with the drug substance, which is completely surrounded by a non-medicated sheath. Accordingly, the release of drug substances from such rings is dependent upon permeation (i.e., molecular dissolution and subsequent diffusion) of the core-loaded drug substance through the outer sheath. Release rates can be modified by changing the thickness of the rate-controlling sheath and/or the length of the drug-loaded core. Reservoir rings were developed to provide controlled (that is, constant daily) release rates. The polymeric materials used in the construction of commercial vaginal rings are typically hydrophobic materials, such as silicone elastomer and poly(ethylene-co-vinyl acetate) (PEVA) materials. Alternatively, polyurethane may be used.

To date, no combination therapy intravaginal rings have been successfully developed. Combining different drugs in the same intravaginal ring poses unique challenges, due to the different solubility and target release rates of different drugs. Another common challenge for intravaginal drug delivery devices is the stability of the device at room temperature for extended time periods. Accordingly, there remains a need for the development of improved intravaginal rings which can be loaded with a combination of antimicrobial drugs to prevent the transmission of HIV, or a combination of an antimicrobial drug and a contraceptive to prevent the transmission of HIV and unintended pregnancy.

### SUMMARY

The present disclosure provides novel combination therapy intravaginal drug delivery devices, i.e., intravaginal rings, useful for the administration of therapeutic and/or prophylactic agents to a human, which solve many of the known challenges with drug release, stability, and storage. For example, the claimed compositions having a specific combination of ethylene-vinyl acetate (EVA) polymers at specific concentrations in the core and the sheath of the ring, and a specific combination of drugs, dapivirine and a contraceptive, such as, levonorgestrol, loaded at specific concentrations into the respective layers of the ring, surprisingly results in intravaginal rings that were not only stable at room temperature, but also were able to provide an independent and controlled release of dapivirine and the contraceptive, over an extended time period, e.g., up to about 12 months or more. In particular, the present disclosure has surprisingly and successfully demonstrated that intravaginal rings, having a core comprising an ethylene-vinyl acetate 28 (EVA 28) polymer and levonorgestrol, and a sheath comprising an ethylene-vinyl acetate 16-25 (EVA 16-25) polymer and dapivirine, at specific concentrations, were able to provide a controlled release of dapivirine and levonorgestrel, over an extended period, e.g., up to about 12 months or more, at room temperature. In addition, the intravaginal rings of the disclosure exhibit an improved mechanical properties and better retention times as compared to other existing intravaginal rings in the art, e.g., silicone rings.

In addition, another challenge in EVA formulations is the instability of dissolved drug in the ring. The intravaginal rings of the present disclosure, however, have further overcome the stability problem by including a higher drug load, which ensures both the adequate drug release over a longer term and a repository of solid drugs remaining undissolved throughout processing to act as seed crystals. As a results, the stability of drugs within the intravaginal rings of the present disclosure are greatly improved.

Accordingly, in one aspect, the disclosure provides an intravaginal ring comprising dapivirine and levonorgestrel, wherein the ring is a reservoir-type ring comprising a core and a sheath; wherein the core comprises an ethylene-vinyl acetate 28 (EVA 28) polymer at a concentration of about 80% to about 95% w/w, and levonorgestrel at a concentration of about 5% to about 20% w/w; wherein the sheath comprises an ethylene-vinyl acetate 16-25 (EVA 16-25) polymer at a concentration of about 70% to about 85% w/w, and dapivirine at a concentration of about 15% to about 30% w/w; and wherein the intravaginal ring has an outer diameter of about 53-57 mm and a cross-sectional diameter of about 3.8-4.5 mm.

In some embodiments, the sheath comprises EVA 16-25 at a concentration of about 80% w/w. In some embodiments, the EVA 16-25 polymer does not comprise an antioxidant.

In some embodiments, the sheath comprises dapivirine at a concentration of about 15-25% w/w.

In some embodiments, the core comprises EVA 28 at a concentration of about 90% w/w. In some embodiments, the EVA 28 polymer does not comprise an antioxidant.

In some embodiments, the core comprises levonorgestrel at a concentration of about 5-15% w/w.

In some embodiments, the intravaginal ring further comprises magnesium stearate. In some embodiments, the magnesium stearate is present at about 0.0%-0.2% w/w in the intravaginal ring.

In some embodiments, levonorgestrel is released from the ring at a zero-order release rate.

In some embodiments, between about 20 μg to about 140 μg of levonorgestrel is released from the intravaginal ring in vitro per day. In some embodiments, between about 40 μg to about 80 μg of levonorgestrel is released from the intravaginal ring in vitro per day. In some embodiments, between about 100 μg to about 140 μg of levonorgestrel is released from the intravaginal ring in vitro per day.

In some embodiments, between about 2500 μg to about 6500 μg of dapivirine is released from the intravaginal ring in vitro during the initial 24 hour period of release.

In some embodiments, between about 300 μg to about 2500 μg of dapivirine is released from the intravaginal ring in vitro per day for about 40 days after the initial 24 hour period of release.

In some embodiments, between about 100 μg to about 600 μg of dapivirine is released from the intravaginal ring in vitro per day for about 50 days after the initial 40-day period of release.

In some embodiments, between about 100 μg to about 600 μg of dapivirine is released from the intravaginal ring in vitro per day for about 140 days after the initial 40-day period of release.

In some embodiments, about 20-140 mg, about 30-120 mg, or about 40-100 mg of dapivirine is present in the sheath. In some embodiments, about 50-350 mg, 100-250 mg, about 110-230 mg, about 120-200 mg, or about 130-180 mg of levonorgestrel is present in the core. In some embodiments, about 20-140 mg, about 30-120 mg, or about 40-100 mg of dapivirine is present in the sheath, and about 50-350 mg, 100-250 mg, about 110-230 mg, about 120-200 mg, or about 130-180 mg of levonorgestrel is present in the core. In some embodiments, about 40-100 mg of dapivirine is present in the sheath, and about 130-180 mg levonorgestrel is present in the core.

In some embodiments, the thickness of the sheath of the ring is about 100-500 μm, about 100-350 μm, or about 100-350 μm. In some embodiments, the thickness of the sheath is about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm.

In some embodiments, the thickness of the sheath is about 120 μm. In some embodiments, the thickness of the sheath is about 240 μm.

In some embodiments, the ring has a weight of about 1.5-2.0 g or about 1.8-2.0 g.

In some embodiments, the hardness of the ring is increased by at least 10%, about 20%, about 30%, about 40%, about 50%, or about 60% as compared to a silicone ring.

In some embodiments, the hardness is determined by measuring the Shore M hardness of the ring and/or the force required to compress the ring over a distance.

In some embodiments, the ring retention time is increased by at least 10%, about 20%, about 30%, about 40%, about 50%, or about 60% as compared to a silicone ring.

In some embodiments, dapivirine is uniformly distributed in the sheath of the ring. In some embodiments, levonorgestrel is uniformly distributed in the core of the ring.

In some embodiments, levonorgestrel is micronized.

In some embodiments, the stability of levonorgestrel in the core of the ring is increased by at least 10%, about 20%, about 30%, about 40%, about 50%, or about 60% as compared to a silicone ring.

In some embodiments, seed crystals of dapivirine are retained in the sheath of the ring. In some embodiments, seed crystals of levonorgestrel are retained in the core of the ring.

In some embodiments, the intravaginal ring is stable at room temperature. In one embodiment, the ring is stable for at least 3 months, at least 4 months, at least 5 months, at least 6 months, at least 7 months, at least 8 months, at least 9 months, at least 10 months, at least 11 months, or at least 12 months.

In one aspect, the present disclosure provides a method of blocking DNA polymerization by an HIV reverse transcriptase enzyme in a female human, comprising the step of inserting the intravaginal ring of the present disclosure into the vagina of the female human.

In another aspect, the present disclosure provides a method of preventing HIV infection in a female human, comprising the step of inserting the intravaginal ring of the present disclosure into the vagina of the female human.

In one aspect, the present disclosure provides a method of treating HIV infection in a female human, comprising the step of inserting the intravaginal ring of the present disclosure into the vagina of the female human.

In another aspect, the present disclosure provides a method of preventing pregnancy and blocking DNA polymerization by an HIV reverse transcriptase enzyme in a female human, comprising the step of inserting the intravaginal ring of the present disclosure into the vagina of the female human.

In one aspect, the present disclosure provides a method of preventing pregnancy and preventing HIV infection in a female human, comprising the step of inserting the intravaginal ring of the present disclosure into the vagina of the female human.

In one aspect, the present disclosure provides a method of preventing pregnancy and treating HIV infection in a female human, comprising the step of inserting the intravaginal ring of the present disclosure into the vagina of the female human.

In one aspect, the present disclosure provides a method of making the intravaginal ring of the present disclosure, the method comprises a) compounding the core comprising the ethylene-vinyl acetate 28 (EVA 28) polymer and dapivirine; b) compounding the sheath comprising the ethylene-vinyl acetate 16-25 (EVA 16-25) polymer and levonorgestrel; c) extruding the core and sheath to form a fiber, d) cutting the fiber to shape into a ring, and e) welding with a heated clamp, thereby making the intravaginal ring.

Other features and advantages of the disclosure will be apparent from the following detailed description and claims.

### BRIEF DESCRIPTION OF THE DRAWINGS

FIG. 1 depicts whole and cross-sectional images of an example DPV-LNG EVA vaginal ring.

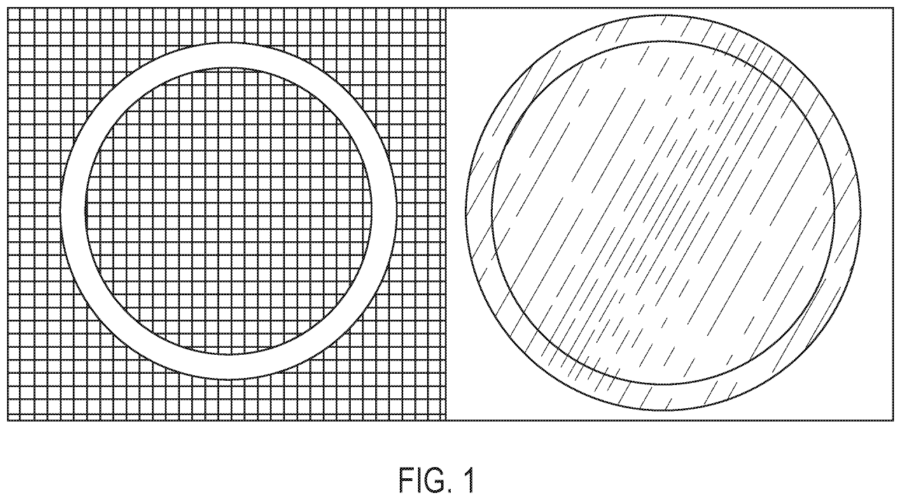

FIG. 2 depicts the molecular structure of DPV.

FIG. 3 depicts the molecular structure of LNG.

FIG. 4 depicts the in vitro release of DPV from an exemplary DPV-LNG EVA vaginal ring.

FIG. 5 depicts the in vitro release of LNG from an exemplary DPV-LNG EVA vaginal ring.

FIG. 6 depicts an overview of DPV-LNG EVA ring manufacturing process.

FIG. 7 depicts the in vitro release profile for DPV from Ring 105 and Ring 106 for up to 93 days of release before (TO) and after 12 months (T12M) of storage at 30° C./75% RH.

FIG. 8 depicts the in vitro release profile for LNG from Ring 105 and Ring 106 for up to 93 days of release before (TO) and after 12 months (T12M) of storage at 30° C./75% RH.

### DETAILED DESCRIPTION

The present disclosure provides novel combination therapy intravaginal drug delivery devices, i.e., intravaginal rings, useful for the administration of therapeutic and/or prophylactic agents to a human, which solve many of the known challenges with drug release, stability, and storage. For example, the claimed compositions having a specific combination of ethylene-vinyl acetate (EVA) polymers at specific concentrations in the core and the sheath of the ring, and a specific combination of drugs, dapivirine and a contraceptive, such as, levonorgestrol, loaded at specific concentrations into the respective layers of the ring, surprisingly results in intravaginal rings that were not only stable at room temperature, but also were able to provide an independent and controlled release of dapivirine and the contraceptive, over an extended time period, e.g., up to about 6 months or more. In particular, the present disclosure has surprisingly and successfully demonstrated that intravaginal rings, having a core comprising an ethylene-vinyl acetate 28 (EVA 28) polymer and levonorgestrol, and a sheath comprising an ethylene-vinyl acetate 16-25 (EVA 16-25) polymer and dapivirine, at specific concentrations, were able to provide a controlled release of dapivirine and levonorgestrel, over an extended period, e.g., up to about 6 months or more, e.g., at room temperature and/or at 37° C. In addition, the intravaginal rings of the disclosure exhibit an improved mechanical properties and better retention times as compared to other existing intravaginal rings in the art, e.g., silicone rings. Another challenge in EVA formulations is the instability of dissolved drug in the ring. The intravaginal rings of the present disclosure, however, have further overcome the stability problem by including a higher drug load, which ensures both the adequate drug release over a longer term and a repository of solid drugs remaining undissolved throughout processing to act as seed crystals. As a results, the stability of drugs within the intravaginal rings of the present disclosure are greatly improved.

### I. Definitions

As used herein, the term “intravaginal ring” or “vaginal ring” refers to a doughnut-shaped or a torus-shaped polymeric drug delivery device which is designed to be inserted into the vagina of a female human in order to provide controlled release of drugs to the vagina over an extended period of time. Several single-indication intravaginal rings are currently available, including Estring® and Femring®, for the treatment of symptoms of post-menopause, and NuvaRing®, a contraceptive vaginal ring.

The intravaginal rings of the instant disclosure provide controlled release of dapivirine, alone or in combination with a contraceptive or an antimicrobial compound, and may have any shape and be of any dimensions compatible with intravaginal administration to a female human. Such a ring can be self-inserted into the vagina, where it is held in place due to its shape and inherent elasticity. Such a device provides high user adherence, case of application and exhibits no leakage or messiness on insertion and subsequent placement within the vaginal space.

As used herein, the term “dapivirine” refers to (4-[[4-[(2,4,6-trimethylphenyl)amino]-2-pyrimidinyl]amino]benzonitrile), a non-nucleoside reverse transcriptase inhibitor. Dapivirine is useful in the prevention and/or treatment of retroviral infection, such as HIV-1 infection. Dapivirine is a crystalline compound that is white to slightly beige in color, has a melting point of about 220° C. and is virtually insoluble in water. More specifically, the solubility of dapivirine is less than 0.001 mg/mLf of water (i.e., less than 1 μg/ml of water). The intravaginal rings of the instant disclosure may use micronized dapivirine. A composite result (four samples taken of micronized material) showed that 88.15% of the material had a particle size of less than 5 microns (μM).

As used herein, the term “antimicrobial compound” or “antimicrobial agent” (used interchangeably herein) refers to a compound or agent which is capable of inhibiting or destroying the growth of a microbial organism. In a preferred embodiment of the disclosure, the antimicrobial compound is a non-nucleoside reverse transcriptase inhibitor (“NNRTI”). In another embodiment, the NNRTI is a substituted di-amino pyrimidine derivative. In another embodiment, the antimicrobial compound is a viral entry inhibitor. In one embodiment, the antimicrobial compound is maraviroc. In one embodiment, the antimicrobial is DS003. In another embodiment of the disclosure, the antimicrobial compound is darunavir, GSK 1265744 or BMS-663068. In another embodiment, the antimicrobial is tenofovir (TNF), or its analogues, e.g., tenofovir disoproxil fumarate (TDF) or tenofovir alafenamide (TAF). The term “antimicrobial compound” is intended to embrace antibacterial agents, antifungal agents, antiprotozoal agents, antiviral agents and mixtures thereof. For purposes of this disclosure, the term “antimicrobial compound” or “antimicrobial agent” is a compound other than dapivirine. That is, the intravaginal rings of the disclosure do not contain only dapivirine as the active agent.

As used herein, the term “contraceptive” refers to an active agent that prevents conception or pregnancy. Contraceptives are well-known in the art and include, but are not limited to, 17α-ethinyl-levonorgestrel-17b-hydroxy-estra-4,9,11-trien-3-one, estradiol, etonogestrel, levonorgestrel, medroxyprogesterone acetate, nestorone, norethindrone, and progesterone. In one embodiment, the contraceptive is levonorgestrel. In one embodiment, the contraceptive is estradiol. In one embodiment, the contraceptive is etonorgestrel. In one embodiment, the contraceptive is nestorone.

As used herein, the term “matrix ring” or “matrix-type ring” refers to an intravaginal ring in which the active agent or agents are homogenously distributed throughout the ring. Matrix rings are typically manufactured by injection molding or extrusion of a compound-containing active mix, leading to the uniform distribution of the active compound throughout the ring. The matrix-type rings of the instant disclosure may comprise dapivirine and either an antimicrobial agent or a contraceptive agent dispersed in silicone elastomer with normal propylorthosilicate (NPOS) crosslinker. This active mix is subsequently cured using a catalyst, such as platinum (with curing achieved by an addition reaction). The matrix-type rings of the disclosure may also comprise polyurethane or ethylene-vinyl acetate (EVA). Matrix-type intravaginal rings permit single intravaginal dosing of active agent(s), with an initially high “loading” and a subsequent, lower “maintenance” release profile.

As used herein, the term “reservoir ring” refers to an intravaginal ring comprising a reservoir (a full or partial-length core), which is completely surrounded by a sheath. In one embodiment, both dapivirine and a contraceptive compound or an antimicrobial compound are present in the core of a reservoir ring, with a blank sheath. In another embodiment, dapivirine and either a contraceptive or an antimicrobial compound are present in separate half-cores (or partial cores) of a reservoir ring, with a blank sheath. In yet another embodiment, either the contraceptive or the antimicrobial compound is present in the core of a reservoir ring, and the dapivirine is present in the sheath. The release of drug substances from such rings is dependent upon permeation (i.e., molecular dissolution and subsequent diffusion) of the core-loaded drug substance through the outer sheath. Release rates can be modified by changing the nature or thickness of the rate-controlling sheath. Reservoir rings were developed to provide controlled (that is, constant daily) release rates.

As used herein, the term “platinum-catalyzed” refers to an intravaginal ring whose cross-linking reaction has been catalyzed using an organo-platinum compound. In one embodiment, the intravaginal ring comprises a silicone elastomer. In yet another embodiment, the intravaginal ring comprises a silicone elastomer and a silicone dispersant. The intravaginal ring may comprise other pharmaceutically compatible agents. Such agents include pharmacologically active agents, as well as, pharmacologically inactive agents known in the art as pharmaceutical excipients.

Various aspects of the disclosure are described in further detail in the following subsections:

### II. Intravaginal Rings

The present disclosure provides combination therapy intravaginal drug delivery devices, i.e., intravaginal rings, useful for the administration of therapeutic and/or prophylactic agents to a human. The intravaginal rings of the disclosure, having a specific combination of ethylene-vinyl acetate (EVA) polymers in the core and the sheath of the ring, were able to provide a controlled release of dapivirine and a contraceptive, such as levonorgestrel, over a period up to about 6 months or more.

As used herein, the term “intravaginal ring” or “vaginal ring” refers to a doughnut-shaped polymeric drug delivery device which is designed to be inserted into the vagina of a female human in order to provide controlled release of drugs to the vagina over an extended period of time. Several single-indication intravaginal rings are currently available, including Estring® and Femring®, for the treatment of urogenital symptoms of post-menopause, and NuvaRing®, a contraceptive vaginal ring. Intravaginal rings are described in U.S. Pat. No. 6,951,654, U.S. Patent Application Publication Nos. US2007/0043332 and US2009/0004246, PCT Publication Nos. WO99/50250, WO02/076426 and WO03/094920, the entire contents of each of which are expressly incorporated herein by reference.

The intravaginal rings of the instant disclosure provide controlled release of dapivirine in combination with either a contraceptive or an antimicrobial compound, and may have any shape and be of any dimensions compatible with intravaginal administration to a female human. Such a ring can be self-inserted into the vagina, where it is held in place due to its shape and inherent elasticity.

In some embodiments, the intravaginal ring has an outer diameter of about 50-60 mm, about 51-59 mm, about 52-5 mm, about 53-57 mm, about 54-56 mm, about 50-55 mm, about 51-56 mm, about 52-57 mm, about 53-58 mm, about 54-59 mm, or about 55-60 mm. In other embodiments, the intravaginal ring has an outer diameter of about 50 mm, about 51 mm, about 52 mm, about 53 mm, about 54 mm, about 55 mm, about 56 mm, about 57 mm, about 58 mm, about 59 mm or about 60 mm.

In some embodiments, the intravaginal ring has a cross-sectional diameter of about 3-8.5 mm, about 3.1-8 mm, about 3.2-7.5 mm, about 3.3-7.0 mm, about 3.4-6.5 mm, about 3.5-6.0 mm, about 3.6-5.5 mm, about 3.7-5.0 mm, or about 3.8-4.5 mm. In other embodiments, the intravaginal ring has a cross-sectional diameter of about 3.0 mm, about 3.1 mm, about 3.2 mm, about 3.3 mm, about 3.4 mm, about 3.5 mm, about 3.6 mm, about 3.7 mm, about 3.8 mm, about 3.9 mm, about 4.0 mm, about 4.1 mm, about 4.2 mm, about 4.3 mm, about 4.4 mm, about 4.5 mm, about 4.6 mm, about 4.7 mm, about 4.8 mm, about 4.9 mm, about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, about 6.1 mm, about 6.2 mm, about 6.3 mm, about 6.4 mm, about 6.5 mm, about 6.6 mm, about 6.7 mm, about 6.8 mm, about 6.9 mm, about 7.0 mm, about 7.1 mm, about 7.2 mm, about 7.3 mm, about 7.4 mm, about 7.5 mm, about 7.6 mm, about 7.7 mm, about 7.8 mm, about 7.9 mm, about 8.0 mm, about 8.1 mm, about 8.2 mm, about 8.3 mm, about 8.4 mm, or about 8.5 mm.

In some embodiments, the thickness of the sheath of the ring is about 100-500 μm, about 100-350 μm, or about 100-350 μm. In some embodiments, the thickness of the sheath is about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, or about 500 μm.

In some embodiments, the thickness of the sheath is about 120 μm. In some embodiments, the thickness of the sheath is about 240 μm.

In some embodiments, the intravaginal ring has a weight of about 1.0-5.0 g, about 1.0-4.0 g, about 1.0-3.0 g, about 1.5-2.0 g or about 1.8-2.0 g. In some embodiments, the intravaginal ring has a weight of about 1.0 g, about 1.1 g, about 1.2 g, about 1.3 g, about 1.4 g, about 1.5 g, about 1.6 g, about 1.7 g, about 1.8 g, about 1.9 g, about 2.0 g, about 2.1 g, about 2.0 g, about 2.1 g, about 2.4 g, about 2.5 g, about 2.6 g, about 2.7 g, about 2.8 g, about 2.9 g, about 3.0 g, about 3.1 g, about 3.2 g, about 3.3 g, about 3.4 g, about 3.5 g, about 3.6 g, about 3.7 g, about 3.8 g, about 3.9 g, about 4.0 g, about 4.1 g, about 4.2 g, about 4.3 g, about 4.4 g, about 4.5 g, about 4.6 g, about 4.7 g, about 4.8 g, about 4.9 g, about 5.0 g.

Such an intravaginal ring permits single intravaginal dosing of dapivirine and either a contraceptive or an antimicrobial agent with a stable release profile. In addition, a device that can be applied less frequently is likely be more acceptable and to achieve better adherence relative to gels that need to be used more frequently

In some embodiments, the intravaginal ring is stable at room temperature. In some embodiments, the intravaginal ring requires no refrigeration.

In one embodiment, the intravaginal ring comprises a silicone elastomer. In yet another embodiment, the intravaginal ring comprises a silicone elastomer and a silicone dispersant. In another embodiment, the intravaginal ring comprises a polyurethane thermoplastic polymer. In yet another embodiment, the intravaginal ring comprises an ethylene-vinyl acetate (EVA) polymer.

The intravaginal ring may comprise other pharmaceutically compatible agents. Such agents include pharmacologically active agents, as well as, pharmacologically inactive agents known in the art as pharmaceutical excipients. Examples of pharmacologically active agents that may be advantageous include, but are not limited to, a local anesthetic such as lidocaine or a local analgesic or a mixture thereof. Examples of pharmacologically inactive agents that may be advantageous include, but are not limited to, a buffer (or buffers), or hydrophilic compounds that enhance the rate of release of the agent from the device, such as for example, polyvinylpyrrolidone (PVP or povidone), modified cellulose ethers (e.g., hydroxyethylcellulose, hydroxypropylcellulose and hydroxypropylmethylcellulose) microcrystalline cellulose, polyacrylic acid, carbomer, alginic acid, carrageenan, cyclodextrins, dextrin, guar gum, gelatin, xanthan gum and sugars (e.g., monosaccharides such as glucose, fructose and galactose, and dissaccharides such as lactose, maltose and fructose). When employed, the release rate enhancing excipient is generally present in an amount of about 0.5 to about 40 w/w % and preferably about 2.5 to about 15 w/w % of the device.

As used herein, the term “matrix ring” or “matrix-type ring” refers to an intravaginal ring in which dapivirine and either an antimicrobial agent or a contraceptive are homogenously distributed throughout the ring. Matrix rings are typically manufactured by injection molding or extrusion of the active compound-containing active mix, leading to the uniform distribution of the active compounds throughout the ring. The matrix-type rings of the instant disclosure may comprise dapivirine and either an antimicrobial compound or a contraceptive dispersed in silicone elastomer with normal propylorthosilicate (NPOS) crosslinker. This active mix is subsequently cured using a catalyst, such as platinum (with curing achieved by an addition reaction). Matrix-type rings may alternatively comprise a polyurethane or EVA polymer.

As used herein, the term “reservoir ring” or “reservoir-type ring” refers to an intravaginal ring comprising a reservoir (a full or partial-length core), which is completely surrounded by a sheath. In one embodiment, either a contraceptive or an antimicrobial compound is present in the sheath of a reservoir ring, and dapivirine is present in the core. In yet another embodiment, either the contraceptive or the antimicrobial compound is present in the core of a reservoir ring, and the dapivirine is present in the sheath. The active agents or drugs, e.g., dapivirine, contraceptive compounds, or antimicrobial compounds, are uniformly distributed in the sheath and/or core of the ring. The release of drug substances from such rings is dependent upon permeation (i.e., molecular dissolution and subsequent diffusion) of the core-loaded drug substance through the outer sheath. Release rates can be modified by changing the nature or thickness of the rate-controlling sheath. Reservoir rings were developed to provide controlled (that is, constant daily) release rates.

In some embodiments, the intravaginal ring of the present disclosure is a reservoir-type ring and comprises dapivirine and a contraceptive, e.g., levonorgestrel. In some embodiments, dapivirine is present in the sheath of the ring, and the contraceptive, e.g., levonorgestrel, is present in the core of the ring.

In some embodiments, dapivirine is present in the sheath of the ring at a concentration of about 1% to about 50% w/w, about 2% to about 45% w/w, about 3% to about 40% w/w, about 4% to about 30% w/w, about 5% to about 20% w/w; about 5% to about 15% w/w, about 10% to about 15% w/w, about 15% to about 25% w/w, or about 15% to about 30% w/w. In some embodiments, dapivirine is present in the sheath of the ring at a concentration of about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, or about 30% w/w.

In some embodiments, a contraceptive, e.g., levonorgestrel, e.g., in a solid state, is present in the core of the ring at a concentration of about 1% to about 50% w/w, about 2% to about 45% w/w, about 3% to about 40% w/w, about 4% to about 30% w/w, about 5% to about 20% w/w; about 5% to about 15% w/w, about 10% to about 15% w/w, about 15% to about 25% w/w, or about 15% to about 30% w/w. In some embodiments, a contraceptive, e.g., levonorgestrel, e.g., in a solid state, is present in the core of the ring at a concentration of about 1% w/w, about 2% w/w, about 3% w/w, about 4% w/w, about 5% w/w, about 6% w/w, about 7% w/w, about 8% w/w, about 9% w/w, about 10% w/w, about 11% w/w, about 12% w/w, about 13% w/w, about 14% w/w, about 15% w/w, about 16% w/w, about 17% w/w, about 18% w/w, about 19% w/w, about 20% w/w, about 21% w/w, about 22% w/w, about 23% w/w, about 24% w/w, about 25% w/w, about 26% w/w, about 27% w/w, about 28% w/w, about 29% w/w, or about 30% w/w.

In some embodiments, the sheath of the ring comprises an ethylene-vinyl acetate polymer, e.g., ethylene-vinyl acetate 16-25 (EVA 16-25) polymer, e.g., EVA16, EVA17, EVA18, EVA19, EVA20, EVA21, EVA22, EVA23, EVA24, or EVA25. In one embodiment, the EVA polymer in the sheath is EVA20. In another embodiment, the EVA polymer in the sheath is EVA24. The EVA polymer in the sheath is at a concentration of about 50% to about 99% w/w, about 60% to about 99% w/w, about 70% to about 99% w/w, about 75% to about 95% w/w, about 80% to about 95% w/w, about 85% to about 95% w/w, about 80% to about 90% w/w, about 70% to about 85% w/w, or about 60% to about 85% w/w. In some embodiments, the EVA polymer in the sheath is at a concentration of about 50% w/w, about 55% w/w, about 60% w/w, about 65% w/w, about 70% w/w, about 75% w/w, about 80% w/w, about 85% w/w, about 90% w/w, about 95% w/w, or about 99% w/w.

In some embodiments, the core of the ring comprises an ethylene-vinyl acetate (EVA) polymer, e.g., ethylene-vinyl acetate 25-30 (EVA 25-30) polymer, e.g., EVA25, EVA26, EVA27, EVA28, EVA29, or EVA30. In one embodiment, the EVA polymer in the core is EVA28. The EVA polymer in the core is at a concentration of about 50% to about 99% w/w, about 60% to about 99% w/w, about 70% to about 99% w/w, about 75% to about 95% w/w, about 80% to about 95% w/w, about 85% to about 95% w/w, about 80% to about 90% w/w, about 70% to about 85% w/w, or about 60% to about 85% w/w. In some embodiments, the EVA polymer in the core is at a concentration of about 50% w/w, about 55% w/w, about 60% w/w, about 65% w/w, about 70% w/w, about 75% w/w, about 80% w/w, about 85% w/w, about 90% w/w, about 95% w/w, or about 99% w/w.

In some embodiments, the EVA polymers comprise an antioxidant. In other embodiments, the EVA polymers do not comprise antioxidants.

The intravaginal ring of the present disclosure may further comprise a lubricant, e.g., magnesium stearate, e.g., at about 0.0%-1.0% w/w, about 0.0%-0.8% w/w, about 0.0%-0.5% w/w or about 0.0%-0.2% w/w in the intravaginal ring. In some embodiments, the intravaginal ring comprises about 0.1% w/w, about 0.2% w/w, about 0.3% w/w, about 0.4% w/w, about 0.5% w/w, about 0.6% w/w, about 0.7% w/w, about 0.8% w/w, about 0.9% w/w, or about 1.0% w/w of magnesium stearate.

The intravaginal rings of the present disclosure exhibit improved mechanical property, e.g., higher Shore M hardness (unitless), or more force required to compress the rings over a certain distance. In some embodiments, the intravaginal rings of the present disclosure are harder than other rings known in the art, e.g., silicone rings such as, Estring, or Femring, by about 10%, about 20%, about 30%, about 40%, about 50%, about 60%, about 70%, about 80% or about 90%. The inventors of the present disclosure compared the Shore M hardness between the intravaginal rings of the present disclosure and other rings known in the art. In particular, the intravaginal ring of the present disclosure was found to have a Shore M hardness of about 82-87, whereas Femring was shown to have a Shore M hardness of about 60, and Estring was shown to have a Shore M hardness of about 54.

Furthermore, the drugs or active agents, e.g., dapivirine, a contraceptive, e.g., levonorgestrel, and/or an antimicrobial agent retained within the intravaginal rings of the present disclosure, exhibit an increased stability when compared to other rings known in the art, e.g., a silicone ring, e.g., platinum catalyzed silicone rings, or tin-catalyzed silicones. A common challenge in EVA formulations is the instability of dissolved drug in the ring. The intravaginal rings of the present disclosure, however, have overcome the problem by including a higher drug load, which ensures both the adequate drug release over a longer term, e.g., 90 days or 180 days, and a repository of solid drugs remaining undissolved throughout processing to act as seed crystals. As a results, the stability of drugs within the intravaginal rings of the present disclosure are greatly improved. In some embodiments, the stability of a contraceptive, e.g., levonorgestrel, in the core of the intravaginal ring of the present disclosure is increased by at least 5%, about 10%, about 15%, about 20%, about 25%, about 30%, about 35%, about 40%, about 45%, or about 50% as compared to other rings known in the art, e.g., a silicone ring. In other embodiments, the stability of dapivirine in the sheath of the intravaginal ring of the present disclosure is increased by at least 5%, about 10%, about 15%, about 20%, about 25%, about 30%, about 35%, about 40%, about 45%, or about 50% as compared to other rings known in the art, e.g., a silicone ring. (See, e.g., PCT Publication No. WO2015086491A1, the entire contents of which are expressly incorporated herein by reference.)

As used herein, the term “elastomer” refers to an amorphous, or predominantly amorphous, polymer network formed when a polymer or a mixture of polymers undergo cross-linking. Each polymer is comprised of monomeric units, which are linked together to form the network. The monomeric units can comprise carbon, hydrogen, oxygen, silicon, halogen, or a combination thereof.

In some embodiments, the intravaginal ring comprises a polysiloxane. As used herein, a “polysiloxane” refers to any of various compounds containing alternate silicon and oxygen atoms in either a linear or cyclic arrangement usually with one or two organic groups attached to each silicon atom. For example, polysiloxanes include substituted polysiloxanes, and diorganopolysiloxanes such as diarylpolysiloxanes and dialkylpolysiloxanes; an example of the latter is dimethylpolysiloxane. Such dimethylpolysiloxane polymers can be thermoset to the corresponding elastomer by vulcanization with peroxide curing catalysts, e.g., benzoyl peroxide or di-p-chlorobenzoyl peroxide at temperatures of about 200° C. and requiring additional heat after treatment as described in U.S. Pat. Nos. 2,541,137; 2,723,966; 2,863,846; 2,890,188; and 3,022,951, the entire contents of each of which are expressly incorporated herein by reference.

An example of a two-component silicone elastomer, which is platinum-catalyzed at room temperature or under slightly elevated temperature and capable of cross-linking, is MED-4870 (NuSil Technology LLC, Carpinteria, CA.). In some embodiments of the present disclosure, an intravaginal ring can comprise silicone liquid (NuSil MED360) as a dispersing agent, and NuSil MED-4870 elastomer. The MED-4870 elastomer is composed of two parts, part A and part B. The chemical composition of MED-4870 part A comprises vinyl terminated polydimethylsiloxane (linear) polymers as a polymer, platinum-siloxane complex as the catalyst for the cross-linking reaction, and ˜30% amorphous (non crystalline) reinforcing silica as a filler. The chemical composition of MED-4870 part B comprises vinyl-terminated polydimethylsiloxane (linear) polymers, hydride functional polydimethysiloxane polymer as a cross-linker, and ˜30% amorphous (non-crystalline) reinforcing silica as a filler. Form A and form B undergo cross-linking to form a silicone elastomer.

In some embodiments of the present disclosure, the polysiloxane elastomer is a diorganopolysiloxane elastomer. In some embodiments, the diorganopolysiloxane elastomer is dimethylpolysiloxane elastomer. In some embodiments, the dimethylpolysiloxane elastomer further comprises a dimethylmethylhydrogen polysiloxane cross-link. In some embodiments of the present disclosure, the polysiloxane elastomer is MED-4870.

In some embodiments, the polysiloxane elastomer is present in a concentration of about 90% to about 99% by total weight of the ring. In some embodiments, the polysiloxane elastomer is present in a concentration of about 95% by total weight of the ring, or about 97% by total weight of the ring.

Suitable cross-linking agents and curing catalysts are well known in the art. Curing temperatures and times will vary, depending on the particular elastomer(s) used. For example, the curing temperature may vary between room temperature (15-25° C.) and 160° C. but is preferably within the range 60-200° C. The curing time may vary between a few seconds and several hours, depending on the elastomer(s) used. Preferred and suitable elastomers include two-component dimethylpolysiloxane compositions using platinum as the curing catalyst and at a curing temperature of from room temperature to an elevated temperature.

As used herein, the term “platinum-catalyzed” refers to an intravaginal ring whose cross-linking reaction has been catalyzed using an organo-platinum compound.

As used herein, the term “alcohol by-product” refers to a volatile by-product of alcohol (including propanol) produced by tin-catalyzed condensation reactions for cross-linking of solid state silicone. Alcohol by-product contributes to an increased rate of migration of antimicrobial compound from within the matrix of an intravaginal ring to the surface, resulting in the undesirable formation of crystalline deposits of antimicrobial compound on the intravaginal ring.

As used herein, the term “crystalline deposits” refers to the undesirable formation of deposits of crystals of dapivirine, the antimicrobial compound or the contraceptive on the surface of the intravaginal ring.

As used herein, the term “release” or “release rate” refers to the amount or concentration of active agent (i.e., dapivirine, or antimicrobial compound or contraceptive) which leaves the intravaginal ring in any defined time period. “Sustained release” or “sustained release rate” refers to release sufficient to provide antimicrobial properties or contraceptive properties over a specific time period. For example, In one embodiment, the intravaginal rings are designed to provide sustained release of dapivirine and either the contraceptive or the antimicrobial compound. In some embodiments, the dapivirine, the contraceptive, or the antimicrobial compound are released from the ring at a zero-order release rate. In other embodiments, the dapivirine, the contraceptive or the antimicrobial compound are released from the ring at a first-order release rate.

As used herein, the term “zero-order release rate” refers to a rate of release of an active agent (i.e., dapivirine, contraceptive or antimicrobial compound) from the ring, which is uniform or nearly uniform independent of the amount or concentration of the active agent during a given time period. In some embodiment, the term “zero-order release rate” also comprises a near zero-order release rate. Therefore, active agents with a zero-order release rate are released from the ring at a constant rate, or a nearly constant rate, that does not change or changes only slightly during the release process.

As used herein, the term “first-order release rate” refers to a rate of release of an active agent (i.e., dapivirine, or contraceptive or antimicrobial compound) from the ring, which is directly proportional to the amount or concentration of the active agent during a given time period. Therefore, changing the concentration or amount of the active agents in the ring also changes the release rate of the active agents.

In some embodiments, the contraceptive, e.g., levonorgestrel, is released from the ring at a zero-order release rate. In some embodiments, about 10 μg to about 200 μg, about 20 μg to about 180 μg, about 20 μg to about 140 μg, about 30 μg to about 100 μg, about 40 μg to about 80 μg, about 40 μg to about 60 μg, or about 40 μg to about 80 μg of levonorgestrel is released from the intravaginal ring in vitro per day. In some embodiments, about 40 μg to about 80 μg of levonorgestrel is released from the intravaginal ring in vitro per day. In some embodiments, about 100 μg to about 140 μg of levonorgestrel is released from the intravaginal ring in vitro per day.

In some embodiments, dapivirine is released from the ring at a first-order release rate. In some embodiments, about 1000 μg to about 8000 μg, about 1500 μg to about 7500 μg, about 2000 μg to about 7000 μg, about 2500 μg to about 6500 μg, or about 3000 μg to about 6000 μg of dapivirine is released from the intravaginal ring in vitro during the initial 24 hour period of release. In some embodiments, about 1000 μg, about 1500 μg, about 1600 μg, about 1700 μg, about 1800 μg, about 1900 μg, about 2000 μg, about 2500 μg, about 3000 μg, about 3500 μg, about 4000 μg, about 4500 μg, about 5000 μg, about 5500 μg, about 6000 μg, about 6500 μg, about 7000 μg, about 7500 μg, or about 8000 μg of dapivirine is released from the intravaginal ring in vitro during the initial 24 hour period of release.

In some embodiments, about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine is released from the intravaginal ring in vitro per day for about 40 days after the initial 24 hour period of release. In some embodiments, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1000 μg, about 1100 μg, about 1200 μg, about 1300 μg, about 1400 μg, about 1500 μg, about 1600 μg, about 1700 μg, about 1800 μg, about 1900 μg, about 2000 μg, about 2100 μg, about 2200 μg, about 2300 μg, about 2400 μg, about 2500 μg, about 2600 μg, about 2700 μg, about 2800 μg, about 2900 μg, or about 3000 μg of dapivirine is released from the intravaginal ring in vitro per day for about 40 days after the initial 24 hour period of release.

In some embodiments, about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine is released from the intravaginal ring in vitro per day for about 50 days after the initial 40-day period of release. In some embodiments, about 50 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of dapivirine is released from the intravaginal ring in vitro per day for about 50 days after the initial 40-day period of release.

In some embodiments, about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine is released from the intravaginal ring in vitro per day for about 140 days after the initial 40-day period of release.

In some embodiments, about 50 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of dapivirine is released from the intravaginal ring in vitro per day for about 140 days after the initial 40-day period of release.

The amount of drug or active agent released from the ring can be determined by any methods or assays known in the art. For example, a release media comprising 0.025 M sodium acetate buffer with 2% Kolliphor HS16 (former tradename Solutol®) can be used. Each ring can be placed in 100 mL release medium and stored in a shaking incubator set at 37° C. and 130 rpm. The release medium is sampled and completely replaced at regular intervals over a total time period. After day 30, the sampling interval may be reduced to twice weekly, on two consecutive days, so that a result for “daily release” (i.e. release in a 24 hour period) could be measured. 100 mL of release media is used for collection every 24 hours. 200 mL of release media is use for longer time intervals.

Values and ranges included and/or intermediate within the ranges set forth herein are also intended to be within the scope of the present disclosure. Ranges having values recited herein as an upper or lower limit are also intended to be within the scope of the present disclosure. Moreover, the amount of dapivirine, antimicrobial compound, or contraceptive released may clearly be varied depending on, for example, the desired dosing level, the particular compound, the release rate effect of excipients used in the device, and the particular elastomeric system employed.

As used herein, the term “initial 24 hour period of use” refers to the first day, or twenty-four hours, of time after the initial use of the intravaginal ring. The initial 24 hour period of use begins when the intravaginal ring is inserted into the vagina of the female human.

As used herein, the term “each day” refers to an individual 24 hour period.

In one embodiment, less than about 2 mg of the antimicrobial compound is released from the ring in vitro during an initial 24 hour period of release. In another embodiment, less than about 1 mg of the antimicrobial compound is released from the ring in vitro during an initial 24 hour period of release.

As used herein, the term “homogenously dispersed throughout” refers to a compound which is uniformly distributed throughout the intravaginal ring.

As used herein, the term “prophylactically effective amount” refers to the amount of antimicrobial compound effective to prevent development of disease in the subject. In one embodiment, the disease is HIV. In a preferred embodiment, a prophylactically effective amount is achieved when between about 1000 μg to about 8000 μg, about 1500 μg to about 7500 μg, about 2000 μg to about 7000 μg, about 2500 μg to about 6500 μg, or about 3000 μg to about 6000 μg of dapivirine and/or antimicrobial compound is released in vitro during an initial 24 hour period of release. In one embodiment, a prophylactically effective amount is achieved when between about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine and/or antimicrobial compound is released from the intravaginal ring in vitro per day for about 40 days after the initial 24 hour period of release. In one embodiment, a prophylactically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 50 days after the initial 40-day period of release. In one embodiment, a prophylactically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 140 days after the initial 40-day period of release.

As used herein, the term “prophylactically effective amount” refers to the amount of contraceptive effective to prevent contraception or pregnancy in the subject. In one embodiment, a prophylactically effective amount is achieved when between about 10 μg to about 200 μg, about 20 μg to about 180 μg, about 20 μg to about 140 μg, about 30 μg to about 100 μg, about 40 μg to about 80 μg, about 40 μg to about 60 μg, or about 40 μg to about 80 μg of levonorgestrel is released from the intravaginal ring in vitro per day.

Values and ranges included and/or intermediate within the ranges set forth herein are also intended to be within the scope of the present disclosure. Ranges having values recited herein as an upper or lower limit are also intended to be within the scope of the present disclosure.

As used herein, the term “therapeutically effective amount” refers to the amount of compound effective to treat disease in the subject. In one embodiment, the disease is HIV. In a preferred embodiment of the disclosure, a therapeutically effective amount is achieved when between about 1000 μg to about 8000 μg, about 1500 μg to about 7500 μg, about 2000 μg to about 7000 μg, about 2500 μg to about 6500 μg, or about 3000 μg to about 6000 μg of dapivirine and/or antimicrobial compound is released in vitro during an initial 24 hour period of release. In one embodiment, a therapeutically effective amount is achieved when between about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine and/or antimicrobial compound is released from the intravaginal ring in vitro per day for about 40 days after the initial 24 hour period of release. In one embodiment, a therapeutically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 50 days after the initial 40-day period of release. In one embodiment, a therapeutically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 140 days after the initial 40-day period of release. In one embodiment, a therapeutically effective amount is achieved when between about 10 μg to about 200 μg, about 20 μg to about 180 μg, about 20 μg to about 140 μg, about 30 μg to about 100 μg, about 40 μg to about 80 μg, about 40 μg to about 60 μg, or about 40 μg to about 80 μg of levonorgestrel is released from the intravaginal ring in vitro per day.

Values and ranges included and/or intermediate within the ranges set forth herein are also intended to be within the scope of the present disclosure. Ranges having values recited herein as an upper or lower limit are also intended to be within the scope of the present disclosure.

As used herein, the term “controlled release rate” refers to a constant release rate that can be determined by the design and drug loading of the vaginal ring.

As used herein, the term “constant release rate” refers to a release rate which does not readily change with device storage over time. Preferably, the release rate of dapivirine, the antimicrobial compound or the contraceptive from the intravaginal ring is constant, or stable and does not readily change over time at room temperature (about 30° C.) or at 40° C. for at least 1 month, at about 2-8° C. for at least 1 year, or for at least 2 years. For example, the release rate of dapivirine, the antimicrobial compound or the contraceptive from the intravaginal rings of the instant disclosure can be stable for 1, 2, 3, 4, 5, 6, 7, 8, 9, 10, 11, 12, 13, 14, 15, 16, 17, 18, 19, 20, 21, 22, 23, 24, 30, 36, 42 or 48 months.

As used herein, the term “steady release rate” means a release rate that shows relatively little change over time.

A “stable” compound is one which essentially retains its physical stability and/or chemical stability and/or biological activity during the manufacturing process and/or upon storage. Various analytical techniques for measuring stability are available in the art and are reviewed in Peptide and Protein Drug Delivery, 247-301, Vincent Lee Ed., Marcel Dekker, Inc., New York, N. Y., Pubs. (1991) and Jones, A. Adv. Drug Delivery Rev. 10:29-90 (1993).

As used herein, the term “storage” refers to the period of time after which the intravaginal rings are made, but before which the intravaginal rings are used. For example, the intravaginal rings of the instant disclosure can be stored for 1, 2, 3, 4, 5, 6, 7, 8, 9, 10, 11, 12, 13, 14, 15, 16, 17, 18, 19, 20, 21, 22, 23, 24, 30, 36, 42 or 48 months.

### III. Dapivirine

The instant disclosure is based, at least in part, on the surprising discovery that intravaginal rings having a specific combination of ethylene-vinyl acetate (EVA) polymers and drugs, e.g., dapivirine, and either an antimicrobial compound or a contraceptive, such as, levonorgestrol, at specific concentrations, in the core and the sheath of the ring are stable, e.g., at room temperature, and provide a long-term controlled release of dapivirine and either an antimicrobial compound or a contraceptive, e.g., levonorgestrel.

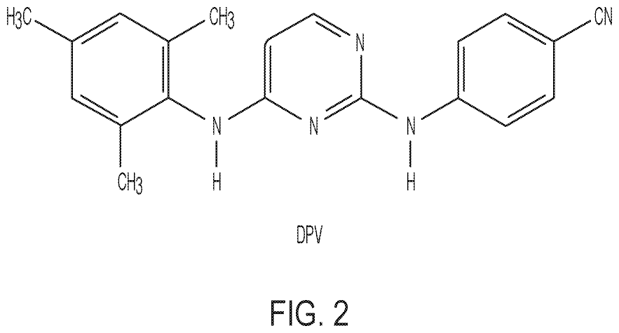

As used herein, the term “dapivirine” refers to (4-[[4-[(2,4,6-trimethylphenyl)amino]-2-pyrimidinyl]amino]benzonitrile), a non-nucleoside reverse transcriptase inhibitor (see structure in FIG. 2).

Dapivirine is useful in the prevention and/or treatment of retroviral infection, such as HIV-1 infection. It is a crystalline compound that is white to slightly beige in color, has a melting point of about 220° C. and is virtually insoluble in water. More specifically, the solubility of dapivirine is less than 0.001 mg/gm of water (i.e., less than 1 μg/ml of water). The intravaginal rings of the instant disclosure may use micronized dapivirine. A composite result (four samples taken of micronized material) showed that 88.15% of the material had a particle size of less than 5 microns (μM).

Dapivirine was originally developed as an oral antiretroviral compound and was first conceived as an oral therapeutic. Dapivirine has potent activity against wild-type HIV-1 strains and HIV-1 strains harboring different resistance-inducing mutations. (Das et al., J. Med Chem., 2004; 47(10):2550-60.) Dapivirine is a white to off-white or slightly yellow powder, free from visible impurities, has a melting point of approximately 220° C., and is virtually insoluble in water. Dapivirine, a substituted DAPY derivate with the chemical name 4-[[4-[(2,4,6-trimethylphenyl)amino]-2-pyrimidinyl]amino]benzonitrile, is a non-nucleoside reverse transcriptase inhibitor (NNRTI).

In some embodiments, about 10 to about 800 mg, about 20 mg to about 140 mg, about 30 mg to about 120 mg, about 40 mg to about 100 mg, about 50 mg to about 750 mg, about 100 mg to about 700 mg, about 10 mg to about 200 mg, about 10 mg to about 150 mg, about 10 mg to about 100 mg, about 50 mg to about 350 mg, about 100 mg to about 200 mg, about 110 mg to about 230 mg, about 120 mg to about 200 mg, about 130 g to about 180 mg, about 150 mg to about 250 mg, about 100 mg to about 250 mg, about 200 mg to about 600 mg, or about 300 mg to about 400 mg of dapivirine is present in the ring, e.g., in the sheath of the ring. In one embodiment, about 20 to about 140 mg of dapivirine is present in the ring, e.g., in the sheath of the ring. In another embodiment, about 30 mg to about 120 mg of dapivirine is present in the ring, e.g., in the sheath of the ring. In another embodiment, about 40 mg to about 100 mg of dapivirine is present in the ring, e.g., in the sheath of the ring.

In one embodiment, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, about 86, about 87 mg, about 88 mg, about 89 mg, about 90 mg, about 95 mg, about 100 mg, about 110 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, or about 800 mg of dapivirine is present in the ring. In another embodiment, about 15 mg of dapivirine is present in the ring. In one embodiment, about 25 mg of dapivirine is present in the ring. In one embodiment, about 45 mg dapivirine is present in the ring. In another embodiment, about 50 mg of dapivirine is present in the ring. In another embodiment, about 80 mg of dapivirine is present in the ring. In another embodiment, about 87 mg of dapivirine is present in the ring. In another embodiment, about 100 mg of dapivirine is present in the ring. In another embodiment, about 150 mg of dapivirine is present in the ring. In another embodiment, about 250 mg of dapivirine is present in the ring.

In one embodiment, the disclosure provides intravaginal rings comprising dapivirine and an antimicrobial compound. In another embodiment, the disclosure provides intravaginal rings comprising dapivirine and a contraceptive. In another embodiment, the disclosure provides intravaginal rings comprising dapivirine, an antimicrobial compound, and a contraceptive.

The intravaginal rings of the disclosure may provide long-term controlled release of dapivirine and an antimicrobial agent, such as maraviroc, DS003, darunavir, GSK1265744, BMS-663068, tenofovir (TNF), or its analogues, e.g., tenofovir disoproxil fumarate (TDF) or tenofovir alafenamide (TAF), or dapivirine and a contraceptive, such as levonorgestrel, estradiol, etonorgestrel or nestorone.

As used herein, the term “release” or “release rate” refers to the amount or concentration of active agent (i.e., dapivirine, or antimicrobial compound or contraceptive) which leaves the intravaginal ring in any defined time period. “Sustained release” or “sustained release rate” refers to release of the active agent sufficient to provide antimicrobial properties or contraceptive properties over a specific time period. For example, In one embodiment, the intravaginal rings are designed to provide sustained release of dapivirine and either the antimicrobial compound or the contraceptive.

In some embodiments, about 1000 μg to about 8000 μg, about 1500 μg to about 7500 μg, about 2000 μg to about 7000 μg, about 2500 μg to about 6500 μg, or about 3000 μg to about 6000 μg of dapivirine is released from the intravaginal ring in vitro during the initial 24 hour period of release.

In some embodiments, about 1000 μg, about 1500 μg, about 1600 μg, about 1700 μg, about 1800 μg, about 1900 μg, about 2000 μg, about 2500 μg, about 3000 μg, about 3500 μg, about 4000 μg, about 4500 μg, about 5000 μg, about 5500 μg, about 6000 μg, about 6500 μg, about 7000 μg, about 7500 μg, or about 8000 μg of dapivirine is released from the intravaginal ring in vitro during the initial 24 hour period of release.

In some embodiments, about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine is released from the intravaginal ring in vitro per day for about 40 days after the initial 24 hour period of release. In some embodiments, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, about 1000 μg, about 1100 μg, about 1200 μg, about 1300 μg, about 1400 μg, about 1500 μg, about 1600 μg, about 1700 μg, about 1800 μg, about 1900 μg, about 2000 μg, about 2100 μg, about 2200 μg, about 2300 μg, about 2400 μg, about 2500 μg, about 2600 μg, about 2700 μg, about 2800 μg, about 2900 μg, or about 3000 μg of dapivirine is released from the intravaginal ring in vitro per day for about 40 days after the initial 24 hour period of release.

In some embodiments, about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine is released from the intravaginal ring in vitro per day for about 50 days after the initial 40-day period of release. In some embodiments, about 50 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of dapivirine is released from the intravaginal ring in vitro per day for about 50 days after the initial 40-day period of release.

In some embodiments, about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine is released from the intravaginal ring in vitro per day for about 140 days after the initial 40-day period of release. In some embodiments, about 50 μg, about 100 μg, about 150 μg, about 200 μg, about 250 μg, about 300 μg, about 350 μg, about 400 μg, about 450 μg, about 500 μg, about 550 μg, about 600 μg, about 650 μg, about 700 μg, about 750 μg, about 800 μg, about 850 μg, about 900 μg, about 950 μg, or about 1000 μg of dapivirine is released from the intravaginal ring in vitro per day for about 140 days after the initial 40-day period of release.

As used herein, the term “increased release” or “increased release rate” refers to the release or the release rate of an active agent (e.g., dapivirine, an antimicrobial compound, or a contraceptive) from an intravaginal ring of the disclosure comprising a combination of dapivirine and either an antimicrobial compound or a contraceptive that is greater, or larger, than the release or the release rate of the active agent (e.g., dapivirine, an antimicrobial compound, or a contraceptive) from an intravaginal ring comprising only one active agent, e.g., dapivirine only, an antimicrobial agent only, or a contraceptive only. Increased release rates can be expressed in terms of increased release per day or cumulative release rate over a period of time, e.g., increased cumulative release over 30 days, over 60 days, over 90 days or 180 days. Increased release rates can be expressed in terms of increased release per day after a prior of time, e.g., increased cumulative release over 20 days, 50 days or 140 days after an initial 40 day period of release. Increased release rates can also be expressed in terms of increased release per day after a period of time, e.g., increased cumulative release over 23 days, 53 days or 83 days after an initial 7 day period of release. Increased release rates can also be expressed in terms of increased release per day after a period of time, e.g., increased cumulative release over 29 days, 59 days, 89 days or 179 days after an initial 24 hour period of release.

In one of the disclosure, the release rate of the dapivirine from a combination therapy intravaginal ring of the disclosure (e.g., a ring comprising dapivirine and either an antimicrobial agent, e.g., maraviroc, or a contraceptive, e.g., levonorgestrel) is not increased as compared to the release rate of dapivirine from an intravaginal ring comprising only dapivirine. In one embodiment, the release rate of the antimicrobial compound or the contraceptive is increased from the combination therapy intravaginal ring of the disclosure as compared to the release rate of the antimicrobial compound or the contraceptive from an intravaginal ring comprising dapivirine without either the antimicrobial compound or the contraceptive.

In another embodiment, the release rate of the dapivirine from a combination therapy intravaginal ring of the disclosure (e.g., a ring comprising dapivirine and either an antimicrobial agent, e.g., maraviroc, or a contraceptive, e.g., levonorgestrel) is increased as compared to the release rate of dapivirine from an intravaginal ring comprising dapivirine without either the antimicrobial compound or the contraceptive. For example, in one embodiment, the release rate of dapivirine from an intravaginal ring of the disclosure comprising dapivirine and either an antimicrobial agent or a contraceptive is increased at least about 2-fold per day as compared to the release rate of dapivirine from an intravaginal ring comprising dapivirine without either the antimicrobial agent or the contraceptive. In one embodiment, the release rate of dapivirine from an intravaginal ring of the disclosure comprising dapivirine and either an antimicrobial agent or a contraceptive is increased at least about 10-fold per day as compared to the release rate of dapivirine from an intravaginal ring comprising dapivirine without either the antimicrobial agent or the contraceptive. In another embodiment, the release rate of dapivirine from an intravaginal ring of the disclosure comprising dapivirine and either an antimicrobial agent or a contraceptive is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold per day as compared to the release rate of dapivirine from an intravaginal ring comprising dapivirine without either the antimicrobial agent or the contraceptive. In one embodiment, the release rate of dapivirine from an intravaginal ring of the disclosure comprising dapivirine and either an antimicrobial agent or a contraceptive is increased by about 2-fold to about 50-fold, by about 2-fold to about 25-fold, by about 2-fold to about 20-fold, by about 2-fold to about 15-fold, by about 2-fold to about 10-fold, by about 2-fold to about 5-fold, by about 5-fold to about 25-fold, by about 5-fold to about 20-fold, by about 5-fold to about 15-fold, by about 5-fold to about 10-fold, by about 10-fold to about 25-fold, or by about 1-fold to about 20-fold per day as compared to the release rate of dapivirine from an intravaginal ring comprising dapivirine without either the antimicrobial agent or the contraceptive.

In one embodiment, the cumulative release rate of dapivirine from an intravaginal ring of the disclosure comprising dapivirine and either an antimicrobial agent or a contraceptive agent is increased at least about 2-fold after 30 days, after 60 days, after 90 days, or after 180 days as compared to the cumulative release rate of dapivirine from an intravaginal ring comprising dapivirine without either the antimicrobial agent or the contraceptive after 30 days, after 60 days, after 90 days, or after 180 days. In one embodiment, the cumulative release rate of dapivirine from an intravaginal ring of the disclosure comprising dapivirine and either an antimicrobial agent or a contraceptive is increased at least about 10-fold after 30 days, after 60 days, after 90 days, or after 180 days as compared to the cumulative release rate of dapivirine from an intravaginal ring comprising dapivirine without either the antimicrobial agent or the contraceptive after 30 days, after 60 days, after 90 days, or after 180 days. In another embodiment, the cumulative release rate of dapivirine from an intravaginal ring of the disclosure comprising dapivirine and either an antimicrobial agent or a contraceptive is increased at least about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45 or 50-fold after 30 days, after 60 days, after 90 days, or after 180 days as compared to the cumulative release rate of dapivirine from an intravaginal ring comprising dapivirine without the antimicrobial agent or the contraceptive after 30 days, after 60 days, after 90 days, or after 180 days. In one embodiment, the cumulative release rate of dapivirine from an intravaginal ring of the disclosure comprising dapivirine and either an antimicrobial agent or a contraceptive is increased by about 2-fold to about 50-fold, by about 2-fold to about 25-fold, by about 2-fold to about 20-fold, by about 2-fold to about 15-fold, by about 2-fold to about 10-fold, by about 2-fold to about 5-fold, by about 5-fold to about 25-fold, by about 5-fold to about 20-fold, by about 5-fold to about 15-fold, by about 5-fold to about 10-fold, by about 10-fold to about 25-fold, or by about 1-fold to about 20-fold after 30 days, after 60 days, after 90 days, or after 180 days as compared to the cumulative release rate of dapivirine from an intravaginal ring comprising dapivirine without the antimicrobial agent or the contraceptive after 30 days, after 60 days, after 90 days, or after 180 days.

Values and ranges included and/or intermediate within the ranges set forth herein are also intended to be within the scope of the present disclosure. Ranges having values recited herein as an upper or lower limit are also intended to be within the scope of the present disclosure.

### IV. Antimicrobial Compounds

The instant disclosure is based, at least in part, on the surprising discovery that intravaginal rings having a specific combination of ethylene-vinyl acetate (EVA) polymers and drugs, e.g., dapivirine, and either an antimicrobial compound or a contraceptive, such as, levonorgestrol, at specific concentrations, in the core and the sheath of the ring are stable, e.g., at room temperature, and provide a long-term controlled release of dapivirine and either an antimicrobial compound or a contraceptive, e.g., levonorgestrel.

As used herein, the term “antimicrobial compound” or “antimicrobial agent” (used interchangeably herein) refers to a compound or agent which is capable of inhibiting or destroying the growth of a microbial organism. In one embodiment, the antimicrobial compound is a non-nucleoside reverse transcriptase inhibitor (“NNRTI”). In one embodiment, the antimicrobial compound is a viral entry inhibitor. In one embodiment, the antimicrobial compound is maraviroc. In one embodiment, the antimicrobial is DS003. In another embodiment of the disclosure, the antimicrobial compound is darunavir, GSK 1265744 or BMS-663068. In another embodiment, the antimicrobial is tenofovir (TNF), or its analogues, e.g., tenofovir disoproxil fumarate (TDF) or tenofovir alafenamide (TAF). The term “antimicrobial compound” is intended to embrace antibacterial agents, antifungal agents, antiprotozoal agents, antiviral agents and mixtures thereof. For purposes of this disclosure, the term “antimicrobial compound” or “antimicrobial agent” is a compound other than dapivirine. That is, the intravaginal rings of the disclosure do not contain only dapivirine as the active agent.

In another embodiment of the disclosure, the antimicrobial compound is a non-nucleoside reverse transcriptase inhibitor (“NNRTI”). In one embodiment, the NNRTI is a substituted di-amino pyrimidine derivative. Useful NNRTI class compounds include, but are not limited to, nevirapine, delavirdine, etravirine and efavirenz. NNRTIs bind to the hydrophobic pocket near the active site of the HIV reverse transcriptase (RT) enzyme, blocking DNA polymerization. (See, e.g., Tarby, Curr. Top. Med. Chem., 2004; 4 (10):1045-57, U.S. Patent Application Publication No. US2006/0166943, and PCT Publication No. WO03/094920, the entire contents of each of which are expressly incorporated herein by reference.) This prevents viral replication and, therefore, production of infectious virus. (Borkow et al., J. Virol., 1997; 71 (4):3023-30.)

In another preferred embodiment of the disclosure, the antimicrobial compound is a viral entry inhibitor. In another embodiment, the viral entry inhibitor is maraviroc. In another preferred embodiment of the disclosure, a nucleoside reverse transcriptase inhibitor is used.

The term “antimicrobial compound” is intended to embrace antibacterial agents, antifungal agents, antiprotozoal agents, antiviral agents and mixtures thereof.

Suitable antibacterial agents include Acrosoxacin, Amifloxacin, Amoxycillin, Ampicillin, Aspoxicillin, Azidocillin, Azithromycin, Aztreonam, Balofloxacin, Benzylpenicillin, Biapenem, Brodimoprim, Cefaclor, Cefadroxil, Cefatrizine, Cefcapene, Cefdinir, Cefetamet, Cefmetazole, Cefprozil, Cefroxadine, Ceftibuten, Cefuroxime, Cephalexin, Cephalonium, Cephaloridine, Cephamandole, Cephazolin,Cephradine, Chlorquinaldol, Chlortetracycline, Ciclacillin, Cinoxacin, Ciprofloxacin, Clarithromycin, Clavulanic Acid, Clindamycin, Clofazimine, Cloxacillin, Danofloxacin, Dapsone, Demeclocycline, Dicloxacillin, Difloxacin, Doxycycline, Enoxacin, Enrofloxacin, Erythromycin, Fleroxacin, Flomoxef, Flucloxacillin, Flumequine, Fosfomycin, Isoniazid, Levofloxacin, Mandelic Acid, Mecillinam, Metronidazole, Minocycline, Mupirocin, Nadifloxacin, Nalidixic Acid, Nifuirtoinol, Nitrofurantoin, Nitroxoline, Norfloxacin, Ofloxacin, Oxytetracycline, Panipenem, Pefloxacin, Phenoxymethylpenicillin, Pipemidic Acid, Piromidic Acid, Pivampicillin, Pivmecillinam, Prulifloxacin, Rufloxacin, Sparfloxacin, Sulbactam, Sulfabenzamide, Sulfacytine, Sulfametopyrazine, Sulphacetamide, Sulphadiazine, Sulphadimidine, Sulphamethizole, Sulphamethoxazole, Sulphanilamide, Sulphasomidine, Sulphathiazole, Temafloxacin, Tetracycline, Tetroxoprim, Tinidazole, Tosufloxacin, Trimethoprim and salts or esters thereof.

Preferred antibacterial agents include tetracyclines such as Doxycycline, Tetracycline or Minocycline; macrolides such as Azithromycin, Clarithromycin and Erythromycin; nitroimidazoles such as Metronidazole or Tinidazole; quinolones such as Ofloxacin, Norfloxacin, Cinoxacin, Ciprofloxacin and Levofloxacin; Clindamycin and Dapsone.

Suitable antifungal agents include Bifonazole, Butoconazole, Chlordantoin, Chlorphenesin, Ciclopirox Olamine, Clotrimazole, Eberconazole, Econazole, Fluconazole, Flutrimazole, Isoconazole, Itraconazole, Ketoconazole, Miconazole, Nifuroxime, Tioconazole, Terconazole, Undecenoic Acid and salts or esters thereof.

Preferred antifungal agents include Clotrimazole, Econazole, Fluconazole, Itraconazole, Ketoconazole, Miconazole, Terconazole and Tioconazole.

Suitable antiprotozoal agents include Acetarsol, Azanidazole, Chloroquine, Metronidazole, Nifuratel, Nimorazole, Omidazole, Propenidazole, Secnidazole, Sineflngin, Tenonitrozole, Temidazole, Tinidazole and salts or esters thereof.

Metronidazole, Tinidazole and Chloroquine are most preferred antiprotozoal agents.

Suitable antiviral agents include Acyclovir, Brivudine, Cidofovir, Curcumin, Dapirivine, Desciclovir, 1-Docosanol, Edoxudine, Fameyclovir, Fiacitabine, Ibacitabine, Imiquimod, Lamivudine, Penciclovir, Valacyclovir, Valganciclovir and salts or esters thereof. Curcumin, Acyclovir, Famcyclovir, Dapirivine and Valacyclovir are preferred antiviral agents.

The most preferred antimicrobial agents of this disclosure include, without limitation, Dapirivine, Metronidazole, Acyclovir, Clotrimazole, Fluconazole, Terconazole, Azithromycin, Erythromycin, Doxycycline, Tetracycline, Minocycline, Clindamycin, Famcyclovir, Valacyclovir, Clarithromycin, a prodrug or salt thereof and combinations thereof.

Mixtures of antibacterial agents, mixtures of antifungal agents; mixtures of antiviral agents; mixtures of antiprotozoal agents and mixtures of agents from two or more of these categories are also envisaged by the present disclosure. In addition, it is also envisaged that the present disclosure embraces at least one antimicrobial agent (microstatic and/or microcidal agent) with one or more other pharmaceutically active agent.

In one embodiment, the intravaginal ring comprises dapivirine and one antimicrobial agent. In another embodiment, the intravaginal ring comprises dapivirine and at least two, at least three, at least four, or at least five antimicrobial agents. In another embodiment, the intravaginal ring comprises dapivirine, a antimicrobial agent, and a contraceptive.

Antimicrobial compounds contained in the rings of the present disclosure are further described at least in U.S. Patent Application Publication Nos. 2012/0093911 and 2006/0166943 and PCT Publication Nos. WO99/50250, WO02/076426 and WO03/094920, the entire contents of each of which are expressly incorporated herein by reference. The antimicrobial compounds contained in the rings of the present disclosure can be prepared according to art-known procedures. In particular, they are prepared according to the procedures described in EP 1002795, WO 99/50250, WO 99/50256 and WO 00/27828, the entire contents of each of which are incorporated herein by reference.

The antimicrobial compounds contained in the rings of the present disclosure may have microbicidal activity and have the ability to prevent the transmission of HIV. In particular, they can prevent sexual or vaginal transmission of HIV by preventing either the production of infectious viral particles or infection of uninfected cells. If infected cells in sperm can reach the mucosa, the compounds of the present disclosure can prevent HIV infection of host cells, such as macrophages, lymphocytes, Langerhans and M cells. Thus, these compounds prevent systemic HIV infection of a human being, exhibiting a prophylactic action against HIV.

In some embodiments, about 10 to about 800 mg, about 20 mg to about 140 mg, about 30 mg to about 120 mg, about 40 mg to about 100 mg, about 50 mg to about 750 mg, about 100 mg to about 700 mg, about 10 mg to about 200 mg, about 10 mg to about 150 mg, about 10 mg to about 100 mg, about 50 mg to about 350 mg, about 100 mg to about 200 mg, about 110 mg to about 230 mg, about 120 mg to about 200 mg, about 130 g to about 180 mg, about 150 mg to about 250 mg, about 100 mg to about 250 mg, about 200 mg to about 600 mg, or about 300 mg to about 400 mg of antimicrobial agent is present in the ring. In one embodiment, about 20 to about 140 mg of antimicrobial agent is present in the ring. In another embodiment, about 30 mg to about 120 mg of antimicrobial agent is present in the ring. In another embodiment, about 40 mg to about 100 mg of antimicrobial agent is present in the ring.

In one embodiment, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 190 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, or about 800 mg of antimicrobial agent is present in the ring. In another embodiment, about 15 mg of antimicrobial agent is present in the ring. In one embodiment, about 25 mg of antimicrobial agent is present in the ring. In another embodiment, about 50 mg of antimicrobial agent is present in the ring. In another embodiment, about 80 mg of antimicrobial agent is present in the ring. In another embodiment, about 100 mg of antimicrobial agent is present in the ring. In another embodiment, about 150 mg of antimicrobial agent is present in the ring. In another embodiment, about 250 mg of antimicrobial agent is present in the ring.

Values and ranges included and/or intermediate within the ranges set forth herein are also intended to be within the scope of the present disclosure. Ranges having values recited herein as an upper or lower limit are also intended to be within the scope of the present disclosure.

### V. Contraceptives

The instant disclosure is based on the surprising discovery that intravaginal rings having a specific combination of ethylene-vinyl acetate (EVA) polymers and drugs, e.g., dapivirine, and either an antimicrobial compound or a contraceptive, such as, levonorgestrol, at specific concentrations, in the core and the sheath of the ring are stable, e.g., at room temperature, and provide a long-term controlled release of dapivirine and either an antimicrobial compound or a contraceptive, e.g., levonorgestrel.

As used herein, the term “contraceptive” refers to an active agent that prevents conception or pregnancy. Contraceptives are well-known in the art and include, but are not limited to, steroid hormones and include, for example, an estrogen, a progestin, a progesterone, a testosterone, derivatives thereof, or combinations thereof. Examples of contraceptives include 17a-ethinyl-levonorgestrel-17b-hydroxy-estra-4,9,11-trien-3-one, estradiol, etonogestrel, levonorgestrel, medroxyprogesterone acetate, nestorone, norethindrone, and progesterone. In one embodiment, the contraceptive is levonorgestrel. In one embodiment, the contraceptive is estradiol. In one embodiment, the contraceptive is etonorgestrel. In one embodiment, the contraceptive is nestorone.

As used herein, an “estrogen” refers to any of various natural or synthetic compounds that stimulate the development of female secondary sex characteristics and promote the growth and maintenance of the female reproductive system, or any other compound that mimics the physiological effect of natural estrogens. Estrogens suitable for use with the present disclosure also include compounds that can be converted to active estrogenic compounds. For example, in some embodiments, a conjugated estrogen can be administered from an intravaginal device of the present disclosure. As used herein, the term “conjugated” refers to the sulfate ester, glucuronide ester, or mixed sulfate-glucuronide esters, of an estrogen. Estrogens suitable for use with the present disclosure also include pharmaceutically suitable salt forms of estrogens. In some embodiments, the salt can be a sodium, potassium, or 2-amino-2-(hydroxymethyl)-1,3-propanediol (Tris) salt. An estrogen suitable for use with the present disclosure can be useful as a contraceptive agent.

Estrogens suitable for use in the present disclosure include, but are not limited to, natural and synthetic compounds having estrogenic activity, such as, for example, estradiol (17β-estradiol), 17α-estradiol, estriol, estrone, and their esters, such as the acetate, sulfate, valerate or benzoate esters of these compounds, including, for example, estradiol 17β-cypionate, estradiol 17-propionate, estradiol 3-benzoate, and piperazine estrone sulfate; ethinyl estradiol; conjugated estrogens (natural and synthetic); agonistic anti-estrogens; and selective estrogen receptor modulators.

Prodrugs of suitable estrogens can also be used in the device of the present disclosure. As used herein, a “prodrug” denotes a derivative of a known direct acting drug, which derivative has enhanced delivery characteristics and therapeutic value as compared to the drug and is transformed into the active drug by an enzymatic or chemical process. Examples of estrogen prodrugs include, but are not limited to, estradiol acetate (which is converted in vivo to 17β-estradiol) and mestranol (which is converted in vivo to ethinyl estradiol). In some embodiments, the estrogen is estradiol, estriol, mestranol, ethinyl estradiol, diethylstilbestrol, or combinations thereof.

As used herein, a “progestin” refers to a progestogen, a progestational substance, or any pharmaceutically acceptable substance in the steroid art that generally possesses progestational activity including synthetic steroids that have progestational activity. Progestins suitable for use with the present disclosure can be of natural or synthetic origin. Progestins generally possess a cyclo-pentanophertanthrene nucleus.

Progestins suitable for use in the present disclosure include, but are not limited to, natural and synthetic compounds having progestational activity, such as, for example, progesterone, medroxyprogesterone, medroxyprogesterone acetate, chlormadinone acetate, norethindrone, cyproterone acetate, norethindrone acetate, desogestrel, levonorgestrel, drospirenone, trimegestone, norgestrel, norgestimate, norelgestromin, etonogestrel, dienogest, gestodene, megestrol, and other natural and/or synthetic gestagens. In some embodiments, the progestin is progesterone, etonogestrel, levonorgestrel, gestodene, norethisterone, drospirenone, or combinations thereof. In one embodiment, the progestin is levonorgestrel. In another embodiment, the progestin is nesterone.

Prodrugs of suitable progestins can also be used in the intravaginal device of the present disclosure. Ethynodiol diacetate, which is converted in vivo to norethindrone, is an example of a progestin prodrug that can be used in the present disclosure. Additional examples of progestin prodrugs include, but are not limited to, norgestimate (which is converted in vivo to 17-deacetyl norgestimate, also known as norelgestromin), desogestrel (which is converted in vivo to 3-keto desogestrel, also known as etonogestrel), and norethindrone acetate (which is converted in vivo to norethindrone).

In some embodiments, the progestin is desogestrel, etonogestrel, norgestimate, or combinations thereof.

In some embodiments, the active agent is a modified testosterone, e.g., a derivative of the synthetic steroid ethisterone. In some embodiments, the modified testosterone is danazol.

In some embodiments, the intravaginal ring of the present disclosure may comprise dapivirine and two contraceptive agents, such as a progestin and an estrogen. In some embodiments, the intravaginal ring comprises dapivirine and a combination of a progestin and an estrogen.

In some embodiments, about 10 to about 800 mg, about 20 mg to about 140 mg, about 30 mg to about 120 mg, about 40 mg to about 100 mg, about 50 mg to about 750 mg, about 100 mg to about 700 mg, about 10 mg to about 200 mg, about 10 mg to about 150 mg, about 10 mg to about 100 mg, about 50 mg to about 350 mg, about 100 mg to about 200 mg, about 110 mg to about 230 mg, about 120 mg to about 200 mg, about 130 g to about 180 mg, about 150 mg to about 250 mg, about 100 mg to about 250 mg, about 200 mg to about 600 mg, or about 300 mg to about 400 mg of contraceptive, e.g., levonorgestrel, is present in the ring, e.g., in the core of the ring. In one embodiment, about 50 to about 350 mg of contraceptive, e.g., levonorgestrel, is present in the ring, e.g., in the core of the ring. In another embodiment, about 100 mg to about 250 mg of contraceptive, e.g., levonorgestrel, is present in the ring, e.g., in the core of the ring. In another embodiment, about 110 mg to about 230 mg of contraceptive, e.g., levonorgestrel, is present in the ring, e.g., in the core of the ring. In another embodiment, about 120 mg to about 200 mg of contraceptive, e.g., levonorgestrel, is present in the ring, e.g., in the core of the ring. In another embodiment, about 130 mg to about 180 mg of contraceptive, e.g., levonorgestrel, is present in the ring, e.g., in the core of the ring.

In one embodiment, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 125 mg, about 130 mg, about 135 mg, about 140 mg, about 145 mg, about 146 mg, about 147 mg, about 148 mg, about 149 mg, about 150 mg, about 155 mg, about 160 mg, about 165 mg, about 166 mg, about 167 mg, about 168 mg, about 169 mg, about 170 mg, about 175 mg, about 180 mg, about 190 mg, about 200 mg, about 225 mg, about 250 mg, about 275 mg, about 300 mg, about 350 mg, about 400 mg, about 450 mg, about 500 mg, about 550 mg, about 600 mg, about 650 mg, about 700 mg, about 750 mg, or about 800 mg of contraceptive, e.g., levonorgestrel, is present in the ring, e.g., in the core of the ring. In another embodiment, about 15 mg of contraceptive, e.g., levonorgestrel, is present in the ring, e.g., in the core of the ring. In one embodiment, about 25 mg of contraceptive, e.g., levonorgestrel, is present in the ring, e.g., in the core of the ring. In another embodiment, about 50 mg of contraceptive, e.g., levonorgestrel, is present in the ring, e.g., in the core of the ring. In another embodiment, about 100 mg of contraceptive, e.g., levonorgestrel, is present in the ring, e.g., in the core of the ring. In one embodiment, about 147 mg of contraceptive, e.g., levonorgestrel, is present in the ring, e.g., in the core of the ring. In another embodiment, about 150 mg of contraceptive, e.g., levonorgestrel, is present in the ring, e.g., in the core of the ring. In one embodiment, about 167 mg of contraceptive, e.g., levonorgestrel, is present in the ring, e.g., in the core of the ring. In another embodiment, about 175 mg of contraceptive, e.g., levonorgestrel, is present in the ring, e.g., in the core of the ring. In another embodiment, about 250 mg of contraceptive, e.g., levonorgestrel, is present in the ring, e.g., in the core of the ring.

Values and ranges included and/or intermediate within the ranges set forth herein are also intended to be within the scope of the present disclosure. Ranges having values recited herein as an upper or lower limit are also intended to be within the scope of the present disclosure.

### VI. Methods for Preventing/Treating HIV and/or Pregnancy

The present disclosure provides methods of preventing and/or treating HIV and preventing pregnancy using the intravaginal rings of the disclosure comprising dapivirine and a contraceptive. The present disclosure also provides methods of preventing and/or treating HIV using the intravaginal rings of the disclosure comprising dapivirine and an antimicrobial compound. In one aspect, the present disclosure provides methods of blocking DNA polymerization by an HIV reverse transcriptase enzyme in a female human, comprising the step of inserting an intravaginal ring of the disclosure into the vagina of the female human. In another aspect, the present disclosure provides methods of preventing HIV infection in a female human, comprising the step of inserting an intravaginal ring of the disclosure into the vagina of the female human. In yet another aspect, the disclosure provides methods of treating HIV infection in a female human, comprising the step of inserting an intravaginal ring of the disclosure into the vagina of the female human. In another aspect, the disclosure provides methods of preventing pregnancy in a female human, comprising the step of inserting an intravaginal ring of the disclosure into the vagina of the female human.

The ring that is inserted into a human may contain a prophylactically effective amount or a therapeutically effective amount of dapivirine and an antimicrobial compound, e.g., maraviroc. The ring that is inserted into a human may contain a prophylactically effective amount or a therapeutically effective amount of dapivirine and a contraceptive, e.g., levonorgestrel.

As used herein, the term “prophylactically effective amount” refers to the amount of dapivirine or the amount of antimicrobial compound effective to prevent development of disease in the subject. In one embodiment, the disease is HIV. In one embodiment, a prophylactically effective amount is achieved when between about 1000 μg to about 8000 μg, about 1500 μg to about 7500 μg, about 2000 μg to about 7000 μg, about 2500 μg to about 6500 μg, or about 3000 μg to about 6000 μg of dapivirine and/or antimicrobial compound is released in vitro during an initial 24 hour period of release. In another embodiment, a prophylactically effective amount is achieved when between about 2500 μg to about 6500 μg of dapivirine and/or antimicrobial compound is released in vitro during an initial 24 hour period of release.

In one embodiment, a prophylactically effective amount is achieved when between about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine and/or antimicrobial compound is released from the intravaginal ring in vitro per day for about 40 days after the initial 24 hour period of release. In another embodiment, a prophylactically effective amount is achieved when between about 100 μg to about 5000 μg, about 300 μg to about 2500 μg of dapivirine and/or antimicrobial compound is released from the intravaginal ring in vitro per day for about 40 days after the initial 24 hour period of release.

In one embodiment, a prophylactically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 50 days after the initial 40-day period of release. In another embodiment, a prophylactically effective amount is achieved when between about 100 μg to about 600 μg of dapivirine and/or antimicrobial compound is released from the intravaginal ring in vitro per day for about 50 days after the initial 40-day period of release.

In one embodiment, a prophylactically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 140 days after the initial 40-day period of release. In another embodiment, a prophylactically effective amount is achieved when between about 100 μg to about 600 μg of dapivirine and/or antimicrobial compound is released from the intravaginal ring in vitro per day for about 140 days after the initial 40-day period of release.

As used herein, the term “prophylactically effective amount” refers to the amount of contraceptive effective to prevent contraception or pregnancy in the subject. In one embodiment, a prophylactically effective amount is achieved when between about 10 μg to about 200 μg, about 20 μg to about 180 μg, about 20 μg to about 140 μg, about 30 μg to about 100 μg, about 40 μg to about 80 μg, about 40 μg to about 60 μg, or about 40 μg to about 80 μg of levonorgestrel is released from the intravaginal ring in vitro per day. In another embodiment of the disclosure, a prophylactically effective amount is achieved when between about 20 μg to about 140 μg of levonorgestrel is released from the intravaginal ring in vitro per day. In one embodiment, a prophylactically effective amount is achieved when between about 40 μg to about 80 μg of levonorgestrel is released from the intravaginal ring in vitro per day.

Values and ranges included and/or intermediate within the ranges set forth herein are also intended to be within the scope of the present disclosure. Ranges having values recited herein as an upper or lower limit are also intended to be within the scope of the present disclosure.

As used herein, the term “therapeutically effective amount” refers to the amount of compound effective to treat disease in the subject. In one embodiment, the disease is HIV. In one embodiment, a therapeutically effective amount is achieved when between about 1000 μg to about 8000 μg, about 1500 μg to about 7500 μg, about 2000 μg to about 7000 μg, about 2500 μg to about 6500 μg, or about 3000 μg to about 6000 μg of dapivirine and/or antimicrobial compound is released in vitro during an initial 24 hour period of release. In another embodiment, a therapeutically effective amount is achieved when between about 2500 μg to about 6500 μg of dapivirine and/or antimicrobial compound is released in vitro during an initial 24 hour period of release.

In one embodiment, a therapeutically effective amount is achieved when between about 100 μg to about 5000 μg, about 150 μg to about 4500 μg, about 250 μg to about 4000 μg, about 300 μg to about 3500 μg, about 350 μg to about 3000 μg, about 300 μg to about 2500 μg, about 400 μg to about 3000 μg, or about 500 μg to about 3500 μg of dapivirine and/or antimicrobial compound is released from the intravaginal ring in vitro per day for about 40 days after the initial 24 hour period of release. In another embodiment, a therapeutically effective amount is achieved when between about 100 μg to about 5000 μg, about 300 μg to about 2500 μg of dapivirine and/or antimicrobial compound is released from the intravaginal ring in vitro per day for about 40 days after the initial 24 hour period of release.

In one embodiment, a therapeutically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 50 days after the initial 40-day period of release. In another embodiment, a therapeutically effective amount is achieved when between about 100 μg to about 600 μg of dapivirine and/or antimicrobial compound is released from the intravaginal ring in vitro per day for about 50 days after the initial 40-day period of release.

In one embodiment, a therapeutically effective amount is achieved when between about 50 μg to about 1000 μg, about 100 μg to about 800 μg, about 100 μg to about 700 μg, about 100 μg to about 600 μg, about 150 μg to about 800 μg, about 200 μg to about 750 μg, about 250 μg to about 700 μg, or about 300 μg to about 650 μg of dapivirine and/or antimicrobial compound is released from the intravaginal ring in vitro per day for about 140 days after the initial 40-day period of release. In another embodiment, a therapeutically effective amount is achieved when between about 100 μg to about 600 μg of dapivirine and/or antimicrobial compound is released from the intravaginal ring in vitro per day for about 140 days after the initial 40-day period of release.

In one embodiment, a therapeutically effective amount is achieved when between about 10 μg to about 200 μg, about 20 μg to about 180 μg, about 20 μg to about 140 μg, about 30 μg to about 100 μg, about 40 μg to about 80 μg, about 40 μg to about 60 μg, or about 40 μg to about 80 μg of levonorgestrel is released from the intravaginal ring in vitro per day. In another embodiment of the disclosure, a therapeutically effective amount is achieved when between about 20 μg to about 140 μg of levonorgestrel is released from the intravaginal ring in vitro per day. In one embodiment, a therapeutically effective amount is achieved when between about 40 μg to about 80 μg of levonorgestrel is released from the intravaginal ring in vitro per day.

Values and ranges included and/or intermediate within the ranges set forth herein are also intended to be within the scope of the present disclosure. Ranges having values recited herein as an upper or lower limit are also intended to be within the scope of the present disclosure.

Those of skill in the prevention and/or treatment of HIV and the prevention of pregnancy could determine the appropriate therapeutically effective amount or prophylactically effective amount from the data presented here in the Examples section. The exact dosage may depend on the particular active agent used.

The term “subject” means female humans who use the rings. Administration of the rings of the present disclosure to a subject can be carried out using known procedures, at dosages and for periods of time effective to treat or prevent HIV or to prevent pregnancy.

As used herein, the term “vagina” or “vaginal” refers to the passage leading from the opening of the vulva to the cervix of the uterus in female humans. As used herein, the term “intravaginal administering” refers to the administration of a ring of the disclosure to the vagina of a female human.

The rings of the present disclosure may be administered into the vagina of a subject prior to sexual intercourse, e.g., 1, 2, 3, 4, 5 or 6 weeks, prior to sexual intercourse. In some embodiments, the rings of the disclosure may be administered into the vagina of a subject after sexual intercourse, e.g., 1, 2, 3, 4, 5, 6, 7, 8, 9, 10, 11, 12, 13, 14 or 15 days after sexual intercourse.

The term sexual intercourse means vaginal sex.

The term “partners” as used herein defines two or more humans, who are sexually active with each other, i.e., who have sexual intercourse with each other.

As used herein, the term “preventing HIV infection” or “preventing HIV transmission” includes the application or administration of an intravaginal ring of the disclosure to a subject who is at risk of developing HIV, or who has been exposed to but not yet developed HIV, in order to decrease the likelihood that the subject will develop HIV. In one embodiment, the term “preventing HIV infection” includes the application or administration of an intravaginal ring of the disclosure to a subject who is at risk of developing HIV, or who has been exposed to but not yet developed HIV, in order to decrease the likelihood that the subject will develop HIV, as compared to a subject who has not been administered an intravaginal ring. In one embodiment, proper use of the intravaginal rings of the disclosure leads to prevention of HIV infection in about 1%, about 5%, about 10%, about 15%, about 20%, about 25%, about 30%, about 35%, about 40%, about 45%, about 50%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% of the subjects who are at risk of developing HIV or who have been exposed to but not yet developed HIV. Values and ranges included and/or intermediate within the ranges set forth herein are also intended to be within the scope of the present disclosure. Ranges having values recited herein as an upper or lower limit are also intended to be within the scope of the present disclosure.

As used herein, the term “preventing pregnancy” includes the application or administration of an intravaginal ring of the disclosure to a subject who is at risk of becoming pregnant in order to decrease the likelihood that the subject will become pregnant. In one embodiment, the term “preventing pregnancy” includes the application or administration of an intravaginal ring of the disclosure to a subject who is at risk of becoming pregnant in order to decrease the likelihood that the subject will become pregnant, as compared to a subject who has not been administered an intravaginal ring. In one embodiment, proper use of the intravaginal rings of the disclosure leads to prevention of pregnancy infection in about 1%, about 5%, about 10%, about 15%, about 20%, about 25%, about 30%, about 35%, about 40%, about 45%, about 50%, about 55%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99% or about 100% of the subjects who are at risk of becoming pregnant. Values and ranges included and/or intermediate within the ranges set forth herein are also intended to be within the scope of the present disclosure. Ranges having values recited herein as an upper or lower limit are also intended to be within the scope of the present disclosure.

The term “treating” includes the application or administration of an intravaginal ring of the disclosure to a subject, or application or administration of an intravaginal ring of the disclosure to a subject who has HIV, with the purpose of curing, healing, alleviating, relieving, altering, remedying, ameliorating, preventing, improving, or affecting HIV. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; or improving a subject's physical or mental well-being. Treatment may be therapeutic or prophylactic. The treatment or amelioration of symptoms can be based on objective or subjective parameters; including the results of a physical examination.

### VII. Methods for Preparing Rings

Intravaginal rings of the disclosure may be manufactured by any method known by those skilled-in-the-art, such as compounding, injection molding, or extrusion.

In some embodiments, the intravaginal ring is a reservoir-type ring comprising a core and a sheath. The ring can be prepared via separately compounding the sheath material (e.g., dapivirine and EVA 20) and the core material (e.g., a contraceptive, e.g., levonorgestrel, and EVA 28). Milled polymer and micronized drug are blended, melt extruded, and pelletized. Subsequently, the core and sheath pellets are separately loaded into co-extruder which simultaneously extrudes both core and sheath material around it forming a fiber. Fibers are then cut to length, shaped into a ring, and welded together with a heated clamp.

Accordingly, the present disclosure provides a method of making an intravaginal ring as describe herein. The method comprises: a) compounding the core comprising the ethylene-vinyl acetate 28 (EVA 28) polymer and dapivirine; b) compounding the sheath comprising the ethylene-vinyl acetate 16-25 (EVA 16-25) polymer and levonorgestrel; c) extruding the core and sheath to form a fiber, d) cutting the fiber to shape into a ring, and e) welding with a heated clamp, thereby making the intravaginal ring.

In some embodiments, the intravaginal rings of the disclosure are prepared by injection molding. The term ‘injection molding’ refers to manufacturing processes for producing parts/devices from either thermoplastic or thermosetting materials using suitably designed injection molds. Examples of thermoplastic materials include polyethylene and PEVA; examples of thermosetting materials include silicone rubbers/elastomers. Without limitation, matrix-type silicone elastomer rings containing dapivirine may be prepared by (i) adding and mixing the dapivirine into one or more components of the silicone system (e.g., base, crosslinking agent, catalyst, excipient, dispersant, etc.) (ii) injecting the mix into suitably designed injection molds, and (iii) optionally, applying heat to cause the silicone mix to cure/crosslink forming an elastomer.

The present disclosure further provides methods of preparing the intravaginal rings of the disclosure described above. These methods generally comprise dispersing dapivirine and either an antimicrobial agent or a contraceptive, and an elastomer, e.g., polysiloxane, in an appropriate solvent or dispersing agent, e.g., silicone liquid, and curing the rings with a platinum catalyst, e.g., a platinum-siloxane complex, thereby preparing a platinum-catalyzed ring. Any of the well-known elastomers, e.g., polysiloxanes, described supra may be used to prepare the platinum-catalyzed rings of the disclosure. In one embodiment, an elastomer, e.g., polysiloxane, for use in the methods of the disclosure is a dimethylsiloxane, e.g., vinyl-terminated polydimethylsiloxane. In another embodiment, an elastomer, e.g., polysiloxane, for use in the methods of the disclosure is a diorganopopolysiloxane, e.g., dimethylpolysiloxane. In another embodiment, the elastomer, e.g., polysiloxane, for use in the methods of the disclosure is MED-8470. In certain embodiments, the methods further comprise use of a cross-linker, e.g., hydride functional polydimethylsiloxane or dimethylmethylhydrogen polysiloxane cross-link.

In one embodiment, the method further comprises catalyzing the rings in a ring mould. The mould can then be opened, following which the intravaginal ring is removed and trimmed. Ring moulds, are preferably coated with, for example, Teflon™ or an electrolytically applied metalised coating. Ring moulds may be constructed of hardened carbon steel, stainless steel, aluminum, or any other material deemed to be appropriate. It will be appreciated that the mould dimensions and design impart the physical shape of the intravaginal drug delivery device, for example, a partial or complete ring, or any other desired shape. Preferably, the device has a partial or complete toroidal shape, more preferably a partial or complete torus shape, or a substantially cylindrical shape. By toroid is meant a ring-like body generated by rotating any closed loop (including an ellipse, a circle or any irregular curve) about a fixed line external to that loop. The toroid shape may be a complete or partial toroid. By torus is meant a ring-like body generated by rotating a circle about a fixed line external to the circle. The torus shape may be a complete or partial ring-like shape. The geometric characteristics of the mould and intravaginal rings can be varied as required by the use.

Alternatively, the intravaginal ring device, or components thereof, may be prepared by extrusional processes, e.g., co-extrusion or blend extrusion, well known to those skilled in the art (see, e.g., U.S. Pat. No. 5,059,363, the entire contents of which are incorporated herein by reference).

The present disclosure is further illustrated by the following examples, which should not be construed as further limiting. The contents of all figures and all references, patents and published patent applications cited throughout this application, as well as the Figures, are expressly incorporated herein by reference in their entirety.

### EXAMPLES

### Example 1: Dapivirine And Levonorgestrel Core-Sheath Ethylene Vinyl Acetate Vaginal Rings

The purpose of this Example was to establish key formulations and process details of a core-sheath ethylene vinyl acetate (EVA) vaginal ring comprising both dapivirine (DAP) and a contraceptive, such as levonorgestrel (LNG), or a DPV-LNG EVA vaginal ring.

The DPV-LNG EVA vaginal ring was a room temperature stable drug/device combination product designed to reduce the risk of HIV acquisition and provide contraception for at least 90 days of continuous use. The core-sheath or reservoir type ring comprised levonorgestrel (LNG) loaded only into the core and dapivirine (DPV) loaded only into the sheath. LNG was released from the ring in a consistent zero order or near zero order pattern in vitro due to being release rate limited by the sheath EVA. DPV was released from the sheath in accordance with first order release kinetics. The core EVA comprised EVA 28 (28% vinyl acetate) and the sheath may comprise EVA with vinyl acetate content between 16% and 25%. The ring was white to off-white in appearance and dimensions were similar to NuvaRing with 4 mm cross-sectional diameter, and a 54 mm outer diameter. Total mass of the DPV-LNG EVA ring was about 1.8-2.0 g. The rings had no perforations, holes, channels, or any other opening through the sheath.

The rings were prepared by first compounding the core and sheath EVAs with LNG or DPV, respectively. Compounded pellets were then smeared with lubricant magnesium stearate and loaded into the hot melt extruder to co-extrude into pellet, cut to length, and welded together at the fiber ends to form a ring.

The DPV-LNG EVA ring was suitable for up to 93 days of continuous use, and had potential for use for up to 12 months or more.

### Active Pharmaceutical Ingredients

### 1. Dapivirine

Dapivirine (DPV) is a di-aminopyrimidine NNRTI that binds to an allosteric site on the reverse transcriptase enzyme, preventing production of proviral DNA and thus replication of the virus. Structure of DPV was shown in FIG. 2. Tables 1 and 2 provide the chemical properties and solubility of DPV in various solvents.

TABLE 1
Chemical properties of DPV

| | |
|---|---|
| Molecular Formula | C20H19N5 |
| Molecular Weight | 329.40 Da |
| Alternative names | TMC-120, R147681, numerous others |
| Chemical name | 4-[[4-[(2,4,6-trimethylphenyl)amino]-2- |
| | pyrimidinyl]amino]benzonitrile |
| CAS registry number | 244767-67-7 |
| Melting point | 220° C. |
| Appearance | White to off-white or slightly yellow powder, |
| | free from visible impurities |
| pKa | 5.8 |
| Log P | 5.27 (pH 9.0) |
| ^{1H} NMR | Consistent with structure. |
| XRPD | Crystalline |
| | Three polymorphs observed, Type I, Type II, |
| | and Type IV |
| DSC | Form I transitions to Form II at 96.9 to 110.3° |
| | C., and Form II melts at 217.9 to 226.9° C. Form |
| | II is not observed at room temperature and |
| | converts to Form I below about 98° C. Form IV is |
| | also stable at room temperature, and converts to |
| | Form II at 209° C. |
| TGA | Molecular decomposition occurs above 250° C. |
| Purity (HPLC) | 98.0 to 102.0% (as is) |

TABLE 2
Solubility of DPV (Form I) in various solvents

| Solvent | Solubility (mg/g) |
|---|---|
| Polyethylene glycol 400 | 47.4 |
| Propylene glycol | 2.69 |
| Water | 0.001 |
| 0.1M HCl | 0.02 |
| 0.01M HCl | 0.17 |
| Buffer pH 2 | 0.095 |
| Buffer pH 4 | 0.0025 |
| Buffer pH 6 | 0.001 |
| Buffer pH 8 | 0.001 |
| Buffer pH 10 | 0.001 |
| 0.1M NaOH | 0.001 |

### 2. Levonorgestrel

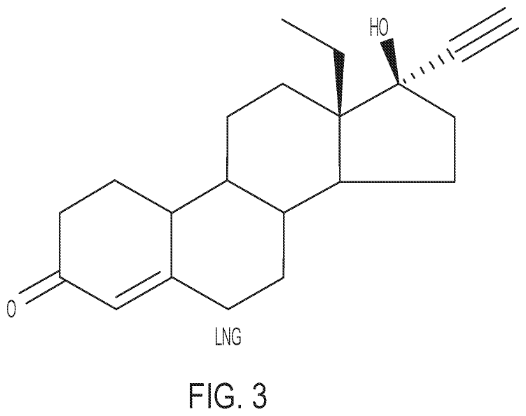

Levonorgestrel (LNG) is a second generation progestin and progesterone receptor agonist. The primary contraceptive mechanism of LNG at the currently approved dose levels is most likely through disruption of the ovulatory cycle, preventing release of the ovum, and prevention of fertilization of the ovum by sperm through changes in cervical mucus. Structure of LNG was shown in FIG. 3. Tables 3 and 4 provide the chemical properties and solubility of LNG in various solvents.

TABLE 3
Chemical properties of LNG

| Molecular Formula | C21H28O2 |
|---|---|
| Molecular Weight | 312.5 Da |
| Alternative names | N/A |
| Chemical name | (17a)-(−)-13-ethyl-17-hydroxy- |
| | 18,19,dinorpregna-4-en-20-yn-3-one |
| CAS registry number | 797-63-7 |
| Melting point | 232-239° C. |
| Appearance | White or almost white crystalline powder |
| ^{1H} NMR | Consistent with structure. No residual |
| | solvent detected |
| XRPD | Crystalline |
| Optical Microscopy | Rectangular shaped crystals ca. 100 × 50 μm |
| DSC | Melt at 240° C. |
| TGA | No weight loss up to 240° C. |
| Karl Fischer(H2O) % w/w | 0.00% w/w (below LOD) |
| Purity (HPLC) | 99.9% |

TABLE 4
Solubility of LNG in various solvents

| | | Solvent | Solubility |
|---|---|---|---|
| Solvent | Weight | Volume | (mg/ml) |
| Water | 12.34 | 4 ml | 0.006 |
| pH = 2, 0.1M USP HCl buffer | 11.06 | 4 ml | 0.002 |
| pH = 4, 0.1M sodium acetate buffer | 17.60 | 4 ml | 0.004 |
| pH = 6, 0.1M sodium acetate buffer | 9.80 | 4 ml | 0.002 |
| pH = 8, 0.1M borate buffer | 11.08 | 4 ml | 0.003 |
| pH = 10, 0.1M borate buffer | 10.68 | 4 ml | 0.001 |
| IPA/water (1:1) | 23.00 | 4 ml | 0.749 |
| Silicone Oil-Dimethylpolysiloxane | 19.02 | 2 ml | 0.008 |
| (Sigma-Aldrich DMPS2X, Mw = ca. | | | |
| 2000) | | | |
| IPA | 20.48 | 2 ml | 2.317 |
| Acetone | 27.04 | 2 ml | 7.150 |
| Ethanol | 20.01 | 2 ml | 4.785 |
| Ethyl Acetate | 19.09 | 2 ml | 4.174 |
| 0.1N HCl with 0.1% SLS (sodium laurel | 19.95 | 2 ml | 0.004 |
| sulfate) | | | |
| 25 mM sodium acetate buffer pH 4.2 | 19.99 | 2 ml | 0.012 |
| with 2% solutol/Kolliphor HS 15 | | | |
| Simulated vaginal fluid, pH 4.2 (without | 5.2 | 2 ml | 0.0049 |
| surfactant) | | | |

### Formulation Details

Table 5 provides the ranges of feasible and target formulation compositions for the DPV-LNG EVA ring.

TABLE 5
Formulation composition of DPV-LNG EVA rings

| | | Feasible |
|---|---|---|
| | Target | concentration |
| | concentration | range |
| Component | (% w/w) | (% w/w) |
| DPV | 20% in sheath | 15-30% in sheath |
| EVA 16-25 | 80% in sheath | 70-85% in sheath |
| LNG | 10% in core | 5-20% in core |
| EVA 28 | 90% in core | 80-95% in core |
| Magnesium | 0.1% in whole | 0.0-0.2% in whole |
| stearate | ring | ring |

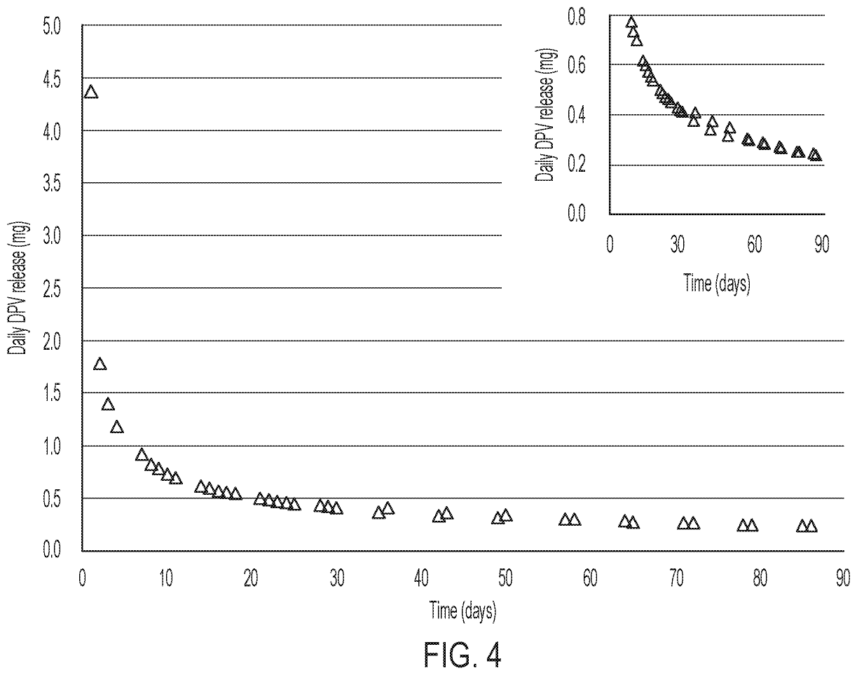

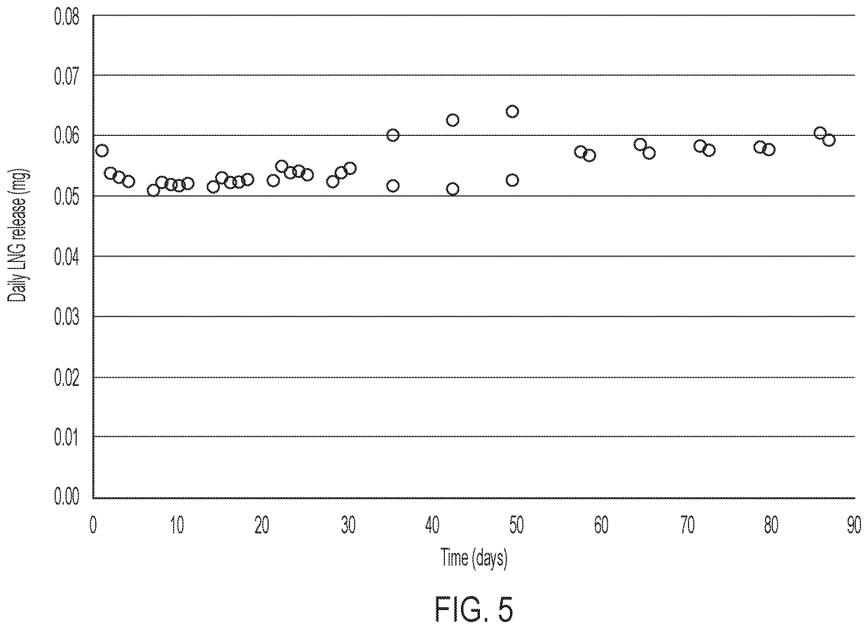

The strategy guiding the formulations in Table 5 focused on achieving target release profiles in vitro and on formulation stability. The vinyl acetate concentration of a given EVA dictates the rate of drug diffusion through the system and generally, the lower the vinyl acetate concentration, the slower DPV or LNG will release from it or migrate through it. Therefore, a lower vinyl acetate material such as EVA 20 was used to obtain a sufficiently slow release of LNG from the EVA 28 core. Due to differences in the properties of the two drugs, DPV released from EVA 20 within the in vitro release target range. An in vitro zero-order release rate range for LNG was approximately 20 to 140 μg/day, and the resulting first-order DPV release Cmax ranged between 2500 and 6500 μg/day, while the release at day 90 ranged from 75-300 μg/day (FIGS. 4 and 5).

A common challenge for EVA formulations was the instability of dissolved drug in polymer as was the reason NuvaRing required refrigeration. One difference between the present intravaginal rings and NuvaRing is the absence of crystalline hormones in NuvaRing. Specifically, in NuvaRing, LNG is present in super saturated state and seed crystals are absent. As a result, the drug persists in dissolved state in a relatively low degree of super saturation. Hence, the system is thermodynamically unstable and on occasion delayed crystallization may take place. Cold storage enhances the kinetic stability of the system. In contrast, in the intravaginal rings of the present disclosure, the drug is assumed to re-crystallize on present seeds resulting in a dissolved concentration equal or close to the saturation concentration.

Indeed, if drug was loaded above the solubility limit in the polymer, or if the system was not heated sufficiently to dissolve all drug, then seed crystals would be retained in the polymer (see, van Laarhoven, Kruft, Vromans. J Control Release. 2002; 82(2-3):309-17).

The specific, custom-manufactured EVA intravaginal rings of the present disclosure were selected based on the target release profiles, but their melt indexes were also suitable for the processing at minimal heat exposures while still being extrudable.

The DPV-LNG EVA ring included a relatively high drug load which both ensured that adequate drug was available for release over 90 days and that a repository of solid drug remains undissolved throughout processing to act as seed crystals. Likewise, prolonged heat exposure was minimized where possible to further promote seed crystal retention.

### Exemplary DPV-LNG EVA Rings

Two EVA rings were designed and prepared as outlined below. Ring 105 contains 87 mg DPV in the sheath and 147 mg LNG in the core of the ring, and has a sheath thickness of 240 μm. Ring 106 contains 45 mg DPV in the sheath and 167 mg LNG in the core of the ering, and has a sheath thickness of 120 μm.

Table 6 provides the stability data for Ring 105 for up to 12 months of storage at 30° C./75% RH and up to 6 months of storage at 40° C./75% RH. Table 7 provides the stability data for Ring 106 for up to 12 months of storage at 30° C./75% RH and up to 6 months of storage at 40° C./75% RH.

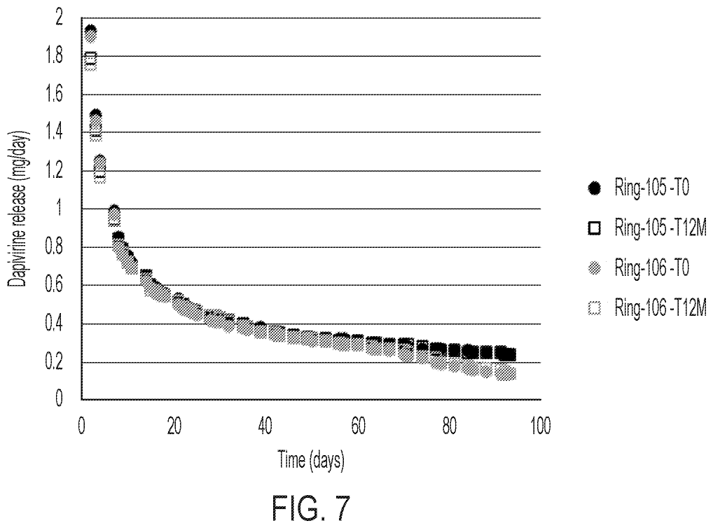

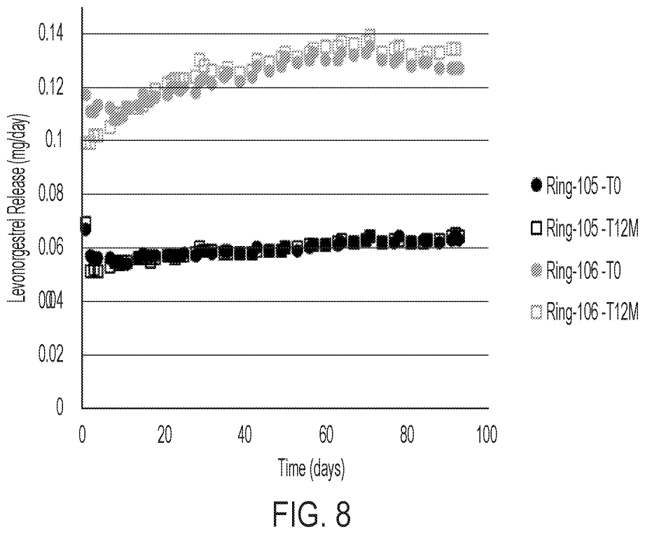

FIG. 7 shows the in vitro release profile for DPV from Ring 105 and Ring 106 for up to 93 days of release before (TO) and after 12 months (T12M) of storage at 30° C./75% RH. FIG. 8 shows the in vitro release profile for LNG from Ring 105 and Ring 106 for up to 93 days of release before (T0) and after 12 months (T12M) of storage at 30° C./75% RH.

These data demonstrate that the DPV-LNG EVA rings are suitable for at least 93 days of continuous use, and also have potential for use for up to 12 months or more.

TABLE 1
Stability data for Ring-105 for up to 12 months of storage at 30°
C./75% RH and up to 6 months of storage at 40° C./75% RH.

| | Storage Condition | | | | | | |
|---|---|---|---|---|---|---|---|
| | | 30° C./75% RH | | | 40° C./75% RH | | |
| | Stability Time Point (months) | | | | | | |
| | 0 | 3 | 6 | 12 | 1 | 3 | 6 |
| Dapivirine Assay (% | 98.8 | 98.4 | 97.9 | 98.8 | 99.7 | 98 | 97.3 |
| of target) | | | | | | | |
| Levonorgestrel Assay | 97.0 | 100.5 | 101.5 | 100.9 | 101.9 | 100 | 101 |
| (% of target) | | | | | | | |
| Ring weight (g) | 1.9 | 1.9 | 1.9 | 1.9 | 1.9 | 1.9 | 1.9 |
| Force at break (N) | 122 | 116 | 118 | 118 | 103 | 112 | 111 |
| Compression Force, 30 | 3.8 | 4.5 | 4.5 | 4.2 | 4.1 | 4.4 | 3.9 |
| mm (N) | | | | | | | |

TABLE 2
Stability data for Ring-106 for up to 12 months of storage at 30°
C./75% RH and up to 6 months of storage at 40° C./75% RH.

| | Storage Condition | | | | | | |
|---|---|---|---|---|---|---|---|
| | | 30° C./75% RH | | | 40° C./75% RH | | |
| | Stability Time Point (months) | | | | | | |
| | 0 | 3 | 6 | 12 | 1 | 3 | 6 |
| Dapivirine Assay (% | 100.2 | 100.4 | 99.1 | 100.9 | 99.4 | 99.8 | 99.6 |
| of target) | | | | | | | |
| Levonorgestrel Assay | 101.7 | 101.4 | 101.8 | 102.4 | 100.5 | 101.2 | 102.6 |
| (% of target) | | | | | | | |
| Ring weight (g) | 1.9 | 1.9 | 1.9 | 1.9 | 1.9 | 1.9 | 1.9 |
| Force at break (N) | 114 | 112 | 111 | 113 | 103 | 112 | 109 |
| Compression Force, 30 | 3.5 | 3.8 | 3.9 | 3.8 | 3.7 | 3.5 | 3.6 |
| mm (N) | | | | | | | |

### Manufacturing Process

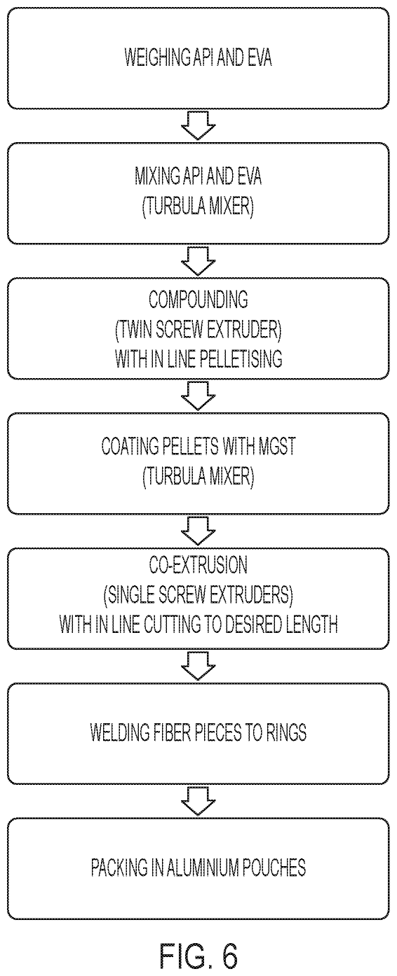

The DPV-LNG EVA ring manufacturing process was outlined in FIG. 6. The process began with blending milled or ground EVAs with their respective drugs in a Turbula mixer. The blend was compounded by hot melt extrusion in a twin screw extruder at 90° C. followed by cutting and pelletization. Pellets were smeared with 0.1% magnesium stearate in the Turbula mixer to lubricate, then the pellets were added to the co-extrusion hoppers for simultaneous extrusion of both the core and sheath ring layers at 80-100° C. and 90-100° C., respectively. The thickness of the core, sheath, and overall ring were dictated by the set speed of the melt pumps which drive the melted material into the dye. The resulting fiber was cut to 157 mm length.

Uniaxial fibers from the extrusion and cutting process were formed into toroid vaginal rings through the use of heat welding without the addition of any further EVA material or adhesive. The welding process warmed the fiber adjacent to the weld while the ends were pressed together in intimate contact allowing the polymer fibers to mesh and align producing a weld with as much tensile strength as the intrinsic fiber strength. The DPV-LNG EVA rings were welded at 110-140° C. (e.g., 120-130° C.) for 10-30 s (e.g., 20-30 s) while the fiber ends were pressed together at 0.5-3.0 kg of horizontal pressure (e.g., 0.5-1.5 kg). The weld process was complete after approximately 30 seconds of cool-down time to return to the 25° C. set point.

### EQUIVALENTS

Those skilled in the art will recognize, or be able to ascertain using no more than routine experimentation, many equivalents to the specific embodiments of the disclosure described herein. Such equivalents are intended to be encompassed by the following claims.

We claim:

1. An intravaginal ring comprising dapivirine and levonorgestrel,
wherein the ring is a reservoir-type ring comprising a core and a sheath;
wherein the core comprises an ethylene-vinyl acetate 28 (EVA 28) polymer at a concentration of about 80% to about 95% w/w, and levonorgestrel at a concentration of about 5% to about 20% w/w;
wherein the sheath comprises an ethylene-vinyl acetate 16-25 (EVA 16-25) polymer at a concentration of about 70% to about 85% w/w, and dapivirine at a concentration of about 15% to about 30% w/w; and
wherein the intravaginal ring has an outer diameter of about 53-57 mm and a cross-sectional diameter of about 3.8-4.5 mm.

2. The intravaginal ring of claim 1, wherein the sheath comprises EVA 16-25 at a concentration of about 80% w/w.

3. The intravaginal ring of claim 1 or 2, wherein the EVA 16-25 polymer does not comprise an antioxidant.

4. The intravaginal ring of any one of the previous claims, wherein the sheath comprises dapivirine at a concentration of about 15-25% w/w.

5. The intravaginal ring of any one of the previous claims, wherein the core comprises EVA 28 at a concentration of about 90% w/w.

6. The intravaginal ring of claim 5, wherein the EVA 28 polymer does not comprise an antioxidant.

7. The intravaginal ring of any one of the previous claims, wherein the core comprises levonorgestrel at a concentration of about 5-15% w/w.

8. The intravaginal ring of any one of the previous claims, further comprising magnesium stearate.

9. The intravaginal ring of claim 8, wherein the magnesium stearate is present at about 0.0%-0.2% w/w in the intravaginal ring.

10. The intravaginal ring of any one of the previous claims, wherein levonorgestrel is released from the ring at a zero-order release rate.

11. The intravaginal ring of any one of the previous claims, wherein between about 20 μg to about 140 μg of levonorgestrel is released from the intravaginal ring in vitro per day.

12. The intravaginal ring of claim 11, wherein between about 40 μg to about 80 μg, or about 100 μg to about 140 μg, of levonorgestrel is released from the intravaginal ring in vitro per day.

13. The intravaginal ring of any one of the previous claims, wherein between about 2500 μg to about 6500 μg of dapivirine is released from the intravaginal ring in vitro during the initial 24 hour period of release.

14. The intravaginal ring of any one of the previous claims, wherein between about 300 μg to about 2500 μg of dapivirine is released from the intravaginal ring in vitro per day for about 40 days after the initial 24 hour period of release.

15. The intravaginal ring of any one of the previous claims, wherein between about 100 μg to about 600 μg of dapivirine is released from the intravaginal ring in vitro per day for about 50 days after the initial 40-day period of release.

16. The intravaginal ring of any one of the previous claims, wherein between about 100 μg to about 600 μg of dapivirine is released from the intravaginal ring in vitro per day for about 140 days after the initial 40-day period of release.

17. The intravaginal ring of any one of the previous claims, wherein about 20-140 mg, about 30-120 mg, or about 40-100 mg of dapivirine is present in the sheath.

18. The intravaginal ring of any one of the previous claims, wherein about 50-350 mg, 100-250 mg, about 110-230 mg, about 120-200 mg, or about 130-180 mg of levonorgestrel is present in the core.

19. The intravaginal ring of any one of the previous claims, wherein the ring has a weight of about 1.5-2.0 g or about 1.8-2.0 g.

20. The intravaginal ring of any one of the previous claims, wherein the thickness of the sheath of the ring is about 100 μm to about 300 μm.

21. The intravaginal ring of any one of the previous claims, wherein the hardness of the ring is increased by at least 10%, about 20%, about 30%, about 40%, about 50%, or about 60% as compared to a silicone ring.

22. The intravaginal ring of claim 21, wherein the hardness is determined by measuring the Shore M hardness of the ring and/or the force required to compress the ring over a distance.

23. The intravaginal ring of any one of the previous claims, wherein the ring retention time is increased by at least 10%, about 20%, about 30%, about 40%, about 50%, or about 60% as compared to a silicone ring.

24. The intravaginal ring of any one of the previous claims, wherein dapivirine is uniformly distributed in the sheath of the ring.

25. The intravaginal ring of any one of the previous claims, wherein levonorgestrel is uniformly distributed in the core of the ring.

26. The intravaginal ring of any one of the previous claims, wherein levonorgestrel is micronized.

27. The intravaginal ring of any one of the previous claims, wherein the stability of levonorgestrel in the core of the ring is increased by at least 10%, about 20%, about 30%, about 40%, about 50%, or about 60% as compared to a silicone ring.

28. The intravaginal ring of any one of the previous claims, wherein seed crystals of dapivirine are retained in the sheath of the ring.

29. The intravaginal ring of any one of the previous claims, wherein seed crystals of levonorgestrel are retained in the core of the ring.

30. The intravaginal ring of any one of the previous claims, wherein the intravaginal ring is stable at room temperature.

31. A method of blocking DNA polymerization by an HIV reverse transcriptase enzyme in a female human, comprising the step of inserting the intravaginal ring of any one of claims 1-30 into the vagina of the female human.

32. A method of preventing HIV infection in a female human, comprising the step of inserting the intravaginal ring of any one of claims 1-30 into the vagina of the female human.

33. A method of treating HIV infection in a female human, comprising the step of inserting the intravaginal ring of any one of claims 1-30 into the vagina of the female human.

34. A method of preventing pregnancy and blocking DNA polymerization by an HIV reverse transcriptase enzyme in a female human, comprising the step of inserting the intravaginal ring of any one of claims 1-30 into the vagina of the female human.

35. A method of preventing pregnancy and preventing HIV infection in a female human, comprising the step of inserting the intravaginal ring of any one of claims 1-30 into the vagina of the female human.

36. A method of preventing pregnancy and treating HIV infection in a female human, comprising the step of inserting the intravaginal ring of any one of claims 1-30 into the vagina of the female human.

37. A method of making the intravaginal ring of any one of claims 1-30, the method comprising
a. compounding the core comprising the ethylene-vinyl acetate 28 (EVA 28) polymer and dapivirine;
b. compounding the sheath comprising the ethylene-vinyl acetate 16-25 (EVA 16-25) polymer and levonorgestrel;
c. extruding the core and sheath to form a fiber,
d. cutting the fiber to shape into a ring, and
e. welding with a heated clamp, thereby making the intravaginal ring.
